SEQUENCE LISTING (8 sequences)

SEQ ID NO: 1 - protein, 661 aa, Homo sapiens [sequence omitted]

SEQ ID NO: 2 - DNA, 3020 bp, Homo sapiens
tggacttgaatggaaggagcccgagcccgcggagcgcagctgagactgggggagcgcgttcggcctgtggggcgccgctcggcgccggggcgcagcaggttccatcagccctggcgcccaggcgcatctgactcggcaccccctgcaggcaccatggcccagagccgggtgctgctgctcctgctgctgctgccgccacagctgcacctgggacctgtgcttgccgtgagggccccaggatttggccgaagtggcggccacagcctgagccccgaagagaacgaatttgcggaggaggagccggtgctggtactgagccctgaggagcccgggcctggcccagccgcggtcagctgcccccgagactgtgcctgttcccaggagggcgtcgtggactgtggcggtattgacctgcgtgagttcccgggggacctgcctgagcacaccaaccacctatctctgcagaacaaccagctggaaaagatctaccctgaggagctctcccggctgcaccggctggagacactgaacctgcaaaacaaccgcctgacttcccgagggctcccagagaaggcgtttgagcatctgaccaacctcaattacctgtacttggccaataacaagctgaccttggcaccccgcttcctgccaaacgccctgatcagtgtggactttgctgccaactatctcaccaagatctatgggctcacctttggccagaagccaaacttgaggtctgtgtacctgcacaacaacaagctggcagacgccgggctgccggacaacatgttcaacggctccagcaacgtcgaggtcctcatcctgtccagcaacttcctgcgccacgtgcccaagcacctgccgcctgccctgtacaagctgcacctcaagaacaacaagctggagaagatccccccgggggccttcagcgagctgagcagcctgcgcgagctatacctgcagaacaactacctgactgacgagggcctggacaacgagaccttctggaagctctccagcctggagtacctggatctgtccagcaacaacctgtctcgggtcccagctgggctgccgcgcagcctggtgctgctgcacttggagaagaacgccatccggagcgtggacgcgaatgtgctgacccccatccgcagcctggagtacctgctgctgcacagcaaccagctgcgggagcagggcatccacccactggccttccagggcctcaagcggttgcacacggtgcacctgtacaacaacgcgctggagcgcgtgcccagtggcctgcctcgccgcgtgcgcaccctcatgatcctgcacaaccagatcacaggcattggccgcgaagactttgccaccacctacttcctggaggagctcaacctcagctacaaccgcatcaccagcccacaggtgcaccgcgacgccttccgcaagctgcgcctgctgcgctcgctggacctgtcgggcaaccggctgcacacgctgccacctgggctgcctcgaaatgtccatgtgctgaaggtcaagcgcaatgagctggctgccttggcacgaggggcgctggcgggcatggctcagctgcgtgagctgtacctcaccagcaaccgactgcgcagccgagccctgggcccccgtgcctgggtggacctcgcccatctgcagctgctggacatcgccgggaatcagctcacagagatccccgaggggctccccgagtcacttgagtacctgtacctgcagaacaacaagattagtgcggtgcccgccaatgccttcgactccacgcccaacctcaaggggatctttctcaggtttaacaagctggctgtgggctccgtggtggacagtgccttccggaggctgaagcacctgcaggtcttggacattgaaggcaacttagagtttggtgacatttccaaggaccgtggccgcttggggaaggaaaaggaggaggaggaagaggaggaggaggaggaagaggaaacaagatagtgacaaggtgatgcagatgtgacctaggatgatggaccgccggactcttttctgcagcacacgcctgtgtgctgtgagccccccactctgccgtgctcacacagacacacccagctgcacacatgaggcatcccacatgacacgggctgacacagtctcatatccccaccccttcccacggcgtgtcccacggccagacacatgcacacacatcacaccctcaaacacccagctcagccacacacaactaccctccaaaccaccacagtctctgtcacacccccactaccgctgccacgccctctgaatcatgcagggaagggtctgcccctgccctggcacgcacaggcacccattccctccccctgctgacatgtgtatgcgtatgcatacacaccacacacacacacatgcacaagtcatgtgcgaacagccctccaaagcctatgccacagacagctcttgccccagccagaatcagccatagcagctcgccgtctgccctgtccatctgtccgtccgttccctggagaagacacaagggtatccatgctctgtggccaggtgcctgccaccctctggaactcacaaaagctggcttttattcctttcccatcctatggggacaggagccttcaggactgctggcctggcctggcccaccctgctcctccaggtgctgggcagtcactctgctaagagtccctccctgccacgccctggcaggacacaggcacttttccaatgggcaagcccagtggaggcaggatgggagagccccctgggtgctgctggggccttggggcaggagtgaagcagaggtgatggggctgggctgagccagggaggaaggacccagctgcacctaggagacacctttgttcttcaggcctgtgggggaagttccgggtgcctttattttttattcttttctaaggaaaaaaatgataaaaatctcaaagctgatttttcttgttatagaaaaactaatataaaagcattatccccaaaaaaaaaaaaaaaaa

SEQ ID NO: 3 - protein, 4 aa, Artificial Sequence
  Description of Artificial Sequence Synthetic peptide
LLNX

SEQ ID NO: 4 - protein, 5 aa, Artificial Sequence
  Description of Artificial Sequence Synthetic peptide
CRPPR

SEQ ID NO: 5 - protein, 9 aa, Artificial Sequence
  Description of Artificial Sequence Synthetic peptide
CPKTRRVPC

SEQ ID NO: 6 - protein, 13 aa, Artificial Sequence
  Description of Artificial Sequence Synthetic peptide
CGFECVRQCPERC

SEQ ID NO: 7 - protein, 4 aa, Artificial Sequence
  Description of Artificial Sequence Synthetic peptide
CEFH

SEQ ID NO: 8 - protein, 4 aa, Artificial Sequence
  Description of Artificial Sequence Synthetic peptide
CEFH

(19) United States
(12) Patent Application Publication
(10) Pub. No.: US 2011/0053852 A1
(43) Pub. Date: Mar. 3, 2011

(54) USE OF PODOCAN PROTEIN IN TREATING CARDIOVASCULAR DISEASES

(76) Inventors: Paul Klotman, New York, NY (US); Randolph Hutter, New York, NY (US); Li Huang, New York, NY (US)

(21) Appl. No.: 12/809,899

(22) PCT Filed: Dec. 19, 2008
(86) PCT No.: PCT/US08/87679

§ 371 Date: Sep. 22, 2010

Related U.S. Application Data
(60) Provisional application No. 61/015,986, filed on Dec. 21, 2007.

(51) Int. Cl. A61K 38/02; A61P 9/12; A61P 9/00

(52) U.S. Cl. 514/15.7; 514/1.1

(57) ABSTRACT

The present invention relates to compositions and methods for the treatment of intimal smooth muscle cell hyperplasia, restenosis following percutaneous coronary intervention, post-transplant vasculopathy, and pulmonary hypertension More particularly, the present invention relates to methods and pharmaceutical compositions for delivering podocan or podocan inhibitors to the arterial system of an animal, thus resulting in the down-regulation or up-regulation, respectively, of smooth muscle cell (SMC) functions such as SMC proliferation and migration Up-regulation of vascular smooth muscle cell proliferation and/or migration by podocan inhibition results in the treatment of vulnerable plaques, while down-regulation of vascular smooth muscle cell proliferation and/or migration via podocan delivery and/or up-regulation results in the treatment of intimal smooth muscle cell hyperplasia, restenosis following percutaneous coronary intervention, post-transplant or graft vasculopathy and pulmonary hypertension.

### CROSS REFERENCE TO RELATED APPLICATIONS

The present application claims priority to U.S. Provisional Application Ser. No. 61/015,986 filed Dec. 21, 2007, which is hereby incorporated by reference in its entirety.

### STATEMENT REGARDING FEDERALLY SPONSORED RESEARCH OR DEVELOPMENT

Research and development leading to certain aspects of the present invention were supported, in part, by a grant from NIH P01DK56492. Accordingly, the U.S. government may have certain rights in the invention.

### FIELD OF THE INVENTION

The present invention relates to compositions and methods for the treatment of intimal smooth muscle cell hyperplasia, restenosis following percutaneous coronary intervention, post-transplant vasculopathy, and pulmonary hypertension using enhancement or blockade of the biologic action of the novel extracellular matrix molecule podocan. Podocan is a novel and selective negative regulator of smooth muscle cell (SMC) function. More particularly, the present invention relates to methods and pharmaceutical compositions for delivering podocan or podocan inhibitors to the arterial system of an animal, thus resulting in the downregulation or upregulation, respectively, of smooth muscle cell (SMC) functions such as proliferation and migration. Upregulation of smooth muscle cell proliferation and/or migration by podocan inhibition results in the treatment of vulnerable plaques stabilizing thin fibrous cap atheroma, while downregulation of vascular smooth muscle cell proliferation and/or migration via podocan delivery and/or upregulation results in the treatment of intimal smooth muscle cell hyperplasia, restenosis following percutaneous coronary intervention, post-transplant or graft vasculopathy and pulmonary hypertension. Podocan is delivered locally to the site of the arterial injury or lesion by means of a stent or delivered systemically, where it binds to collagen-exposed and de-endothelialized portions of the luminal surface of the arterial wall.

### BACKGROUND OF THE INVENTION

### The Biology of Arterial Lesions

Arterial lesion formation represents a vascular response to injury that results from diverse noxious stimuli ranging from local mechanical (endothelial denudation, balloon angioplasty, and stenting) to systemic/metabolic triggers (hypertension, hyperlipidemia, hyperglycemia, immunologic injury).^{1,2,3} Advanced cardiovascular disease is characterized by the presence of multiple arterial lesions throughout the arterial side of the vascular tree. Dependent on location, size, and thrombogenicity, these lesions determine a wide array of clinical events in patients ranging from acute coronary syndrome, cerebro-vascular events and peripheral arterial disease, to name just a few key clinical manifestations of atherosclerotic disease. (Cardiovascular disease describes the overall clinical entity of various disease manifestations in patients caused by the underlying pathological substrate/process in the arterial system which is called atherosclerosis. Both terms are often used somewhat synonymously in medical literature (Ross R. Atherosclerosis—an inflammatory disease. N Engl J Med 1999; 340:115-26; Newby A C, Zaltsman A B. Molecular mechanisms in intimal hyperplasia. J. Pathol. 2000; 190:300-9). Each type of arterial lesion is comprised mainly of three different major cell types (endothelial cells, vascular smooth muscle cells, and inflammatory cells).

Endothelial cells cover the luminal endovascular surface, sealing highly thrombogenic extracellular-matrix components and pro-thrombotic cellular material accumulating in atherosclerotic lesions from the bloodstream and thus preventing the onset of acute or subacute arterial thrombosis.

Vascular smooth muscle cells comprise the medial layer of the healthy arterial wall. Only in response to any form of arterial injury these cells are found in the intimal space and contribute to arterial lesion progression. Smooth muscle cells (SMCs) play a dual role in arterial lesion progression. In vasculo-proliferative lesions marked by a high degree of intimal vascular smooth muscle cell hyperplasia—meaning accumulation of excessive amounts of intimal smooth muscle cells—smooth muscle cells contribute to gradual luminal narrowing by growing into a bulk of intimal tissue that ultimately impedes arterial blood flow. This process, however, occurs over a long period of time lasting up to several years in highly stenotic primary lesions or lasting up to several months in restenotic lesions post-percutaneous coronary intervention (Hoffmann R, Mintz G S, Dussaillant G R, Popma J J, Pichard A D, Satler L F, Kent K M, Griffin J, Leon M B. Patterns and mechanisms of in-stent restenosis. A serial intravascular ultrasound study. Circulation. 1996; 94:1247-54; Schwartz R S, Huber K C, Murphy J G, Edwards W D, Camrud A R, Vlietstra R E, Holmes D R. Restenosis and the proportional neointimal response to coronary artery injury: results in a porcine model. J Am Coll Cardiol. 1992; 19:267-74).

In contrast, vulnerable plaques comprise a significant amount of inflammatory cells (predominantly macrophages), are comparably devoid of smooth muscle cells and initially are only mildly stenotic. Vulnerable plaques are small, lipid-rich plaques that are rich in inflammatory cells and are only covered by a thin fibrous cap with deceased SMC activity and accelerated matrix degradation. Therefore, thinning of the fibrous cap and rupture is a consequence of insufficient SMC activity. This knowledge is described by autopsy and angiographic studies. In these plaques, SMCs provide the fibrous cap covering the highly thrombogenic inflammatory cell content in a similar fashion as endothelial cells prevent this material from interacting with the arterial bloodstream. Vulnerable plaques become highly symptomatic when the fibrous cap comprised of smooth muscle cells ruptures; acute arterial thrombosis ensues, resulting in an often instantaneous occlusion of arterial blood flow (Hoffman et al. and Schwartz et al., supra. Seventy-five percent of myocardial infarctions are thought to be caused by a vulnerable plaque rupture event causing subsequent thrombosis.

### Current Standard of Care for Non-Surgical Treatment of Arterial Lesions

Current treatment regimes for stenosis or occluded vessels include mechanical interventions. However, these techniques also serve to exacerbate the injury, precipitating new smooth muscle cell proliferation and neointimal growth. The standard of care for the non-surgical treatment of arterial blockages caused by both, vasculo-proliferative lesions and vulnerable plaques, is to re-open the blockage with an angioplasty balloon, often followed by the placement of a wire metal structure called a stent to retain the opening in the artery. The effectiveness of this procedure is limited in some patients because the treatment itself damages the vessel, thereby inducing proliferation of smooth muscle and reocclusion or restenosis of the vessel. It has been estimated that approximately 30 to 40 percent of patients treated by balloon angioplasty and/or stents may experience restenosis within one year of the procedure. This number has been lowered by the use of current DES (DES restenosis rate is about 10 to 15%).

In order to address the issue of restenosis post-percutaneous coronary intervention (PCI), drug-eluting stents (DESs) have been developed and currently represent about 60% of all stents employed. These DESs act by non-specifically blocking cell proliferation by eluting non-specific, pro-apoptotic compositions such as paclitaxel or rapamycin. The non-SMC-selective and non-physiologic anti-proliferative and pro-apoptotic strategies employed by current DESs effectively inhibit restenosis, however, this is achieved at the expense of a delay in luminal endothelial repair and with pro-coagulant side effects requiring prolonged anti-platelet therapy and carrying the small but clinically very significant risk of late in-stent thrombosis (mortality of more than 50% if it occurs).^{38,39}

Therefore, specific and selective modulation of arterial smooth muscle cell activity would allow navigating the relatively small therapeutic window of excessive (e.g., restenosis post-PCI) versus insufficient SMC activity (vulnerable plaque), without resulting in the disadvantages and risks associated with the non-specific nature of current drug-eluting stents (e.g., delay of reendothelialization of the site of the plaque).

### The Biology of Post-Transplant Vascular Disease

Graft vasculopathy (GVP) (also known as post-transplant vascular disease or post-transplant vasculopathy) is the major threat to the long-term survival of cardiac allograft recipients and consists in the development of diffuse intimal thickening in the allograft coronary arteries through mechanisms that are poorly understood (Billingham M E. Graft coronary disease: the lesions and the patients. Transplant Proc. 1989; 21:3665-6; Costanzo M R, Naftel D C, Pritzker M R, Heilman J K, 3rd, Boehmer J P, Brozena S C, Dec G W, Ventura H O, Kirklin J K, Bourge R C, Miller L W. Heart transplant coronary artery disease detected by coronary angiography: a multi-institutional study of preoperative donor and recipient risk factors. Cardiac Transplant Research Database. J Heart Lung Transplant. 1998; 17:744-53; Tullius S G, Tilney N L. Both alloantigen-dependent and -independent factors influence chronic allograft rejection. Transplantation. 1995; 59:313-8). GVP also remains a major obstacle to the long-term success of renal and lung allografts (Tullius S G, Tilney N L. Both alloantigen-dependent and -independent factors influence chronic allograft rejection. Transplantation. 1995; 59:313-8; Shi C, Lee W S, He Q, Zhang D, Fletcher D L, Jr., Newell J B, Haber E. Immunologic basis of transplant-associated arteriosclerosis. Proc Natl Acad Sci USA. 1996; 93:4051-6). Strategies to control GVP traditionally have focused on modulation of lymphocyte stimulation with limited success (Zerbe T, Uretsky B, Kormos R, Armitage J, Wolyn T, Griffith B, Hardesty R, Duquesnoy R. Graft atherosclerosis: effects of cellular rejection and human lymphocyte antigen. J Heart Lung Transplant. 1992; 11:S104-10). Syndromes of accelerated atherogenesis all morphologically display a profound and clinically relevant intimal hyperplasia (IH) (transplant vascular disease, veins used as arterial bypass conduits or arteriovenous fistulae, and post-angioplasty and stent restenosis). Each of these conditions is a patho-physiologic scenario where intimal SMC hyperplasia occurs. (Hayry P, Paavonen T, Mennander A, Ustinov J, Raisanen A, Lemstrom K. Pathophysiology of allograft arteriosclerosis. Transplant Proc. 1993; 25:2070; Libby P, Salomon R N, Payne D D, Schoen F J, Pober J S. Functions of vascular wall cells related to development of transplantation-associated coronary arteriosclerosis. Transplant Proc. 1989; 21:3677-84). The development of IH is associated with an increase in the number of smooth muscle cells (SMC) and the extracellular matrix. This process occurs in response to injury to the vascular wall whether that injury be alloimmune, traumatic, ischemic or hemodynamic (Shi C, Lee W S, He Q, Zhang D, Fletcher D L, Jr., Newell J B, Haber E. Immunologic basis of transplant-associated arteriosclerosis. Proc Natl Acad Sci USA. 1996; 93:4051-6; Libby P, Salomon R N, Payne D D, Schoen F J, Pober J S. Functions of vascular wall cells related to development of transplantation-associated coronary arteriosclerosis. Transplant Proc. 1989; 21:3677-84; Newby A C, Zaltsman A B. Molecular mechanisms in intimal hyperplasia. J Pathol. 2000; 190:300-9; Ross R. Atherosclerosis—an inflammatory disease. N Engl J Med. 1999; 340:115-26).

In the context of cardiac transplantation, GVP is characterized by a diffuse concentric intimal proliferation of SMC with preservation of the IEL. Only much later in the process of GVP do lipid-containing cells and cholesterol clefts appear in a segmental fashion, with lesions very similar to those encountered in atherosclerotic plaques with cell death events and secondary thrombosis. The whole process is exclusively limited to the allograft and its progression is significantly faster in comparison with the rate of native atherosclerosis progression (Shi C, Lee W S, He Q, Zhang D, Fletcher D L, Jr., Newell J B, Haber E. Immunologic basis of transplant-associated arteriosclerosis. Proc Natl Acad Sci USA. 1996; 93:4051-6; Newby A C, Zaltsman A B. Molecular mechanisms in intimal hyperplasia. J Pathol. 2000; 190:300-9; Shi C, Russell M E, Bianchi C, Newell J B, Haber E. Murine model of accelerated transplant arteriosclerosis. Circ Res. 1994; 75:199-207; Soleimani B, Katopodis A, Wieczorek G, George A J, Hornick P I, Heusser C. Smooth muscle cell proliferation but not neointimal formation is dependent on alloantibody in a murine model of intimal hyperplasia. Clin Exp Immunol. 2006; 146:509-17).

Although the pathological changes typical in GVP have been well defined, the underlying etiology and origin of the disease remains unclear. However, given the role of SMC hyperproliferation in GVP, a selective inhibitor of SMCs, capable of normalizing SMC function and proliferation, would be an instrumental and novel method to treat GVP.

### The Biology of Pulmonary Hypertension

Pulmonary arterial hypertension (PAH) is characterized by selective elevation of pulmonary arterial pressure. The pathological hallmark of PAH is the narrowing of pulmonary arterioles secondary to endothelial dysfunction and smooth muscle cell proliferation (Rubin L J. Primary pulmonary hypertension. N Engl J Med. 1997; 336:111-7; Humbert M, Sitbon O, Simonneau G. Treatment of pulmonary arterial hypertension. N Engl J. Med. 2004; 351:1425-36).

PAH is a progressive and ultimately fatal disease defined by selective elevation of the mean pulmonary arterial pressure by at least 25 mmHg at rest or >30 mmHg during exercise (Rubin et al. and Humbert et al., supra). The underlying cause of this sustained elevation is an increased pulmonary vascular resistance, resulting in progressive right heart hypertrophy, reduced right heart function, and heart failure caused by increased right ventricular afterload (Rubin L J. Primary pulmonary hypertension. N Engl J. Med. 1997; 336:111-7; Rich S, Dantzker D R, Ayres S M, Bergofsky E H, Brundage B H, Detre K M, Fishman A P, Goldring R M, Groves B M, Koerner S K, et al. Primary pulmonary hypertension. A national prospective study. Ann Intern Med. 1987; 107:216-23; Newman J H. Pulmonary hypertension. Am J Respir Grit Care Med. 2005; 172:1072-7). A key event in the development of PAH is pulmonary vascular remodeling, a complex process involving all layers and cells of the vessel wall (including endothelial and smooth muscle cells as well as adventitial fibroblasts) (Pietra G G, Capron F, Stewart S, Leone O, Humbert M, Robbins I M, Reid L M, Tuder R M. Pathologic assessment of vasculopathies in pulmonary hypertension. J Am Coll Cardiol. 2004; 43:25 S-32S; Meyrick B. The pathology of pulmonary artery hypertension. Clin Chest Med. 2001; 22:393-404, vii); Hopkins N, McLoughlin P. The structural basis of pulmonary hypertension in chronic lung disease: remodelling, rarefaction or angiogenesis? J. Anat. 2002; 201:335-48; Stenmark K R, Fagan K A, Frid M G. Hypoxia-induced pulmonary vascular remodeling: cellular and molecular mechanisms. Circ Res. 2006; 99:675-91). Structural changes that are observed routinely in PAH include smooth muscle cell hyperplasia and increased deposition of extra-cellular matrix proteins (including collagen and elastin).

Although the pathological changes typical in PAH have been well defined, the underlying etiology and origin of the disease remains unclear. However, given the role of SMC hyperproliferation in PAH, a selective inhibitor of smooth muscle cells, capable of normalizing SMC function and proliferation, would be an instrumental and novel method to treat PAH.

Thus, the selective inhibitor of smooth muscle cells disclosed in the present invention can be employed in the treatment of pulmonary hypertension. Such an inhibitor can be administered systemically or locally via, for example, a catheter inserted into the right-hear pulmonary artery.

### Podocan

Analogous to inflammatory and fibrotic processes in renal interstitial and glomerular disease, collagen vascular diseases, and disorders of bone metabolism, the vascular production and remodeling of extracellular matrix (ECM) has a profound regulatory role on the key repair cells involved⁴⁻⁸. A family of small ECM proteins defined by a leucin-repeat rich core protein and different GAG-side chains is especially potent in modulating cellular phenotype⁹. This growing family of small leucine-rich proteoglycan proteins (SLRP) comprises up to now IV classes defined by the number of leucine-rich repeats (LRRs), the N-terminal composition, and their number of exons. Among these molecules members of class I, Biglycan and Decorin, are the best studied ECM cellular effector molecules significantly modulating such complex pathological processes as fibrosis and cancer growth⁹⁻¹⁴. Podocan is a recently discovered member of the SLRP family differing in all three classifying categories and, therefore, establishes a new (fifth) class of this protein family. Podocan was identified by representational differential analysis of cDNA in HIV-1 transgenic and non-transgenic podocytes¹⁵. Podocan mRNA and protein expression has been observed at strongly increased levels in sclerotic glomerular lesions of HIV-associated nephropathy (HIVAN) but has also been seen in normal heart, kidney and in SMC in vitro¹⁶.

To test the effect of podocan on SMC function and arterial repair in vivo mice genetically deficient in podocan (podocan^{-/-} (knockout) mice) were generated (Hutter et al., Evidence for Increased Neointima Formation in Podocan Knock-out Mice compared to C57/BL6 Wild Type Mice after Arterial Denudating Injury; The Mount Sinai Journal of Medicine, (May 2006) Vol. 73, No. 3, 637; Hutter et al., Evidence for Increased Neointima Formation in Podocan Knock-out Mice compared to C57/BL6 Wild Type Mice after Arterial Denudating Injury; oral presentation at American Heart Association conference Nov. 15, 2006). An established femoral arterial denudating injury model that yields SMC-rich neointimal lesions when performed under normo-lipidemic conditions was applied to these podocan^{-/-} mice. In addition, primary SMC cultures from explanted aorta with either podocan^{-/-} or WT genotype were generated to further characterize a possible altered podocan−/− SMC phenotype in vitro and to test if the WT phenotype can be rescued by transfection of podocan−/− SMC with podocan gene.

The present invention provides for the specific and selective modulation of SMC activity via modulation of podocan expression and activity. Podocan protein shows a distinct expression pattern in human coronary restenotic plaques versus vulnerable plaques; podocan is expressed predominantly intracellularly in restenotic lesions and is expressed and deposited predominantly extracellularly in vulnerable plaques. The in vivo and in vitro data presented herewith derived from the podocan−/− mouse model demonstrates that the delivery of podocan to restenotic lesions and the delivery of podocan-blocking molecules to vulnerable lesions will selectively downregulate and upregulate, respectively, smooth muscle cell density/numbers in different types of arterial lesions, thus treating these lesions without disrupting reendothelialization. Similarly, podocan can be used to treat transplant vasculopathy and pulmonary hypertension. Thus, the present invention provides for the specific and selective modulation of SMC activity, a goal of cardiovascular disease treatment that has remained until now an elusive goal.

### SUMMARY OF THE INVENTION

The instant invention provides methods of treating occlusion of a body vessel which comprises administering podocan or a functional equivalent that regulates smooth muscle cell activity. In a preferred embodiment, the podocan or the functional equivalent thereof is linked to or embedded in a matrix or other peptide/protein. In another preferred embodiment the body vessel is a blood vessel. In other preferred embodiments the body vessel body vessel is selected from the group consisting of the artery, vein, common bile duct, pancreatic duct, kidney duct, esophagus, trachea, urethra, bladder, uterus, ovarian duct, fallopian tube, vas deferens, prostatic duct, or lymphatic duct.

In yet another preferred embodiment the smooth muscle cell activity is smooth muscle cell proliferation or smooth muscle cell migration. In yet another preferred embodiment the occlusion is caused by a condition selected from the group consisting of atherosclerosis, restenosis of a blood vessel, transplant vasculopathy, vein-graft atherosclerosis, thrombosis, angioplasty restenosis, and pulmonary hypertension.

One embodiment of the methods of the invention is administering locally, including locally injecting podocan or locally administering podocan or the functional equivalent by placing a medical or biocompatible device coated with podocan protein or its functional equivalent at the site of the occlusion or locally administering podocan or the functional equivalent by placing a medical or biocompatible device coated with a nucleic acid encoding podocan or its functional equivalent at the site of the occlusion.

The instant invention provides methods of treating occlusion of a body vessel, wherein the condition comprises pulmonary hypertension and podocan or a functional equivalent is administered through a right-heart luminal device in the pulmonary artery. In a preferred embodiment the medical or biocompatible device is an intraluminal device, and, more particularly, the intraluminal device is selected from the group consisting of a stent, a wire, a catheter, or a sheath.

In another embodiment of the invention, the podocan or the functional equivalent is administered in combination with a compound that inhibits proliferation of smooth muscle cells, wherein the compound can include paclitaxel, rapamycin, actinomycin D, or radioactivity.

The instant invention also provides methods for diagnosing atherosclerosis in a patient, which comprises (i) obtaining a sample from said patient, (ii) measuring an expression level of podocan in said sample (iii) comparing said expression level with a standard, and wherein an increase in the level of podocan as compared to the podocan standard indicates atherosclerosis. In a preferred embodiment the measuring step comprises determining the expression level of podocan polypeptide or podocan mRNA. In a yet another embodiment, the podocan or the functional equivalent is provided with a cell penetrating peptide, preferably a homing peptide.

The instant invention also provides for intraluminal devices coated with a nucleic acid encoding podocan or a functional equivalent of podocan or coated with a podocan polypeptide or a functional equivalent of said polypeptide. In a preferred embodiment, the intraluminal device is selected from the group consisting of a stent, a wire, a catheter, or a sheath. In a further preferred embodiment, the intraluminal device is further coated with collagen or a collagen matrix or the podocan is embedded in the collagen or collagen matrix.

The instant invention also provides for methods of treating occlusion of a body vessel by administering an agent that regulates smooth muscle cell activity, wherein said agent is a podocan inhibitor or a functional equivalent thereof. In one embodiment, the occlusion comprises a vulnerable plaque. In yet another embodiment, the inhibitor is a member selected from the group consisting of a podocan antisense oligonucleotide, a podocan-specific RNAi construct, a podocan antibody or a small molecule inhibitor of podocan.

The instant invention also provides for method of inhibiting smooth muscle cell proliferation which comprises contacting a smooth muscle cell with podocan or a functional equivalent thereof, whereby proliferation of said smooth muscle cell is inhibited. In a preferred embodiment, the smooth muscle cell comprises a vascular smooth muscle cell. In a further preferred embodiment, the contacting comprises administering to a site at risk of undesired smooth muscle cell proliferation a cell growth inhibitory amount of podocan or a functional equivalent thereof, whereby a smooth muscle cell proliferative disorder is treated. In yet a further embodiment the contacting comprises administering to a patient at risk of restenosis an effective amount of podocan or a functional equivalent thereof for inhibiting vascular smooth muscle cell proliferation.

In yet another aspect of the invention, the effective amount of podocan or a functional equivalent thereof is administered to said patient before, during or after an angioplasty procedure. In a further preferred embodiment, the administering includes delivering podocan or a functional equivalent thereof to an angioplasty site in said patient. In yet a further embodiment, the stent is a drug-eluting stent capable of releasing podocan or a functional equivalent thereof in situ.

The methods of the instant invention can be, for example, applied a patient at risk of atherosclerosis progression whereby the risk of atherosclerosis progression in the patient is treated, to a patient at risk of keloid formation, to a patient suffering from cancer originating from a smooth muscle cell, whereby proliferation of a cancer cell is inhibited.

These and other aspects of the present invention will become apparent upon reference to the following detailed description and attached drawings. All publications, patents, and patent applications cited herein, whether supra or infra, are hereby incorporated by reference in their entirety.

### BRIEF DESCRIPTION OF THE DRAWINGS

FIG. 1 A-H. FIG. 1 shows the immunohistochemical staining for podocan in mouse femoral artery and human atheroma (A-D lower power and E-H high power magnification images). (A, E) Blue shows DAPI-staining (i.e., location of nuclei), while brown shows podocan staining. Podocan staining is absent in non-injured wild-type femoral arteries, ×400, ×1000; (B, F) Distinct podocan deposition in the intra- as well as extracellular space is seen in response to femoral artery denudating injury indicating expression of podocan in medial and neointimal SMC at four weeks after arterial injury; ×400, ×1000; (C, G) In injured femoral artery of podocan−/− mice podocan staining is completely absent indicating that no podocan expression has occurred in the abundant SMC accumulating in the intimal space; ×400, ×1000. (D, H) In areas of plaque repair in human carotid atheroma marked by neovascularization and cellular infiltrates podocan signals are strongly present in the extracellular space; ×200, ×1000.

FIG. 2 A-F. FIG. 2 shows immunofluorescence double-labeling for smooth muscle alpha-actin and podocan in injured wild-type femoral artery two weeks after arterial injury (A-C low power and D-F high power images):

(A, D) Medial alpha-actin expression and nascent intimal alpha-actin expression is seen by red fluorescent (Texas Red) signals with a luminal predominance of alpha-actin expression in differentiating SMC. ×400, ×1000. (B, E) Selective green fluorescent labeling (FITC) indicating the presence of podocan is seen in the cytoplasm of a majority of medial and intimal cells but is absent in adventitial cells; ×400, ×1000. (C, F) Overlay indicates podocan expression that precedes alpha-actin expression in not yet fully differentiated intimal SMC at two weeks. ×400, ×1000.

FIG. 3 A-G. FIG. 3 shows the comparison of neointima formation in podocan WT and podocan−/− groups at one, two and four weeks after arterial injury. FIGS. 3A-F show the immunohistochemical staining of femoral artery cross sections demonstrating the time course of arterial response to injury at one (A, D), two (B, E) and four (C, F) week after injury comparing wild type (A-C) and podocan^{-/-} genotype (D-F) as seen with Masson's trichrome stain: (A, D) At one week early intimal cell adhesion can be seen concomitant to an adventitial cellular infiltrate; in both vascular spaces cellularity appears somewhat higher with podocan^{-/-} genotype; ×400. (B, E) At two weeks a dense and highly cellular intimal lesion has formed preceding the phase of extensive intimal ECM secretion and remodeling typically occurring between two and four weeks in this model; at this time only a trend towards a larger intimal lesion with but no significant difference in arterial lesion size is seen; ×400. (C, F) At four weeks a mildly stenotic arterial lesion has formed in injured arteries of WT mice; in contrast, with podocan^{-/-} genotype a dramatically enhanced arterial lesion has formed that is increased in size as well as cellular content; ×400. FIG. 3G is a bar graph showing the comparison of neointima formation in podocan WT and podocan^{-/-} groups at one, two and four weeks after arterial injury: Neointima area in ×10-² mm² (independent sample t-test).

FIG. 4 A-G. FIG. 4 shows the time course of arterial response to injury at one, two, and four week time points in wild type and podocan−/− mice as seen with anti-smooth muscle alpha-actin immunostaining: (A, D) At one week alpha-actin expression is predominantly seen in the medial compartment in both groups; the adventitial cellular infiltrate is largely alpha-actin negative; ×400. (B, E) At two weeks a nascent and predominantly luminal alpha-actin expression is observed in an early intimal lesion with high cell density; at this time the adventitial cellular infiltrate along the EEL in the podocan^{-/-} group partially expresses alpha-actin; a trend towards higher arterial lesion SMC density is seen with the podocan^{-/-} genotype; ×400. (C, F) At four weeks the arterial lesion has matured and remodeled with expression of alpha-actin in nearly all intimal cells. In the absence of podocan a significant increase in SMC density is observed late after initial injury; ×400. FIG. 4G is a bar graph showing the comparison of neointimal SMC density as assessed by alpha-actin expression in podocan WT and podocan^{-/-} groups at one, two and four weeks after arterial injury: Cell density in ×10³ mm² (independent sample t-test).

FIG. 5 A-G. FIG. 5 A-F shows the time course of cell proliferation during arterial response to injury showing one (A, D), two (B, E) and four (C, F) week time points comparing wild type (A-C) and podocan^{-/-} genotype (D-F) as seen with Ki-67 and alpha-actin immunofluorescence double-labeling: (A, D) At one week select and distinct medial and adventitial proliferative signals are found at a comparable level in both groups; ×400. (B, E) At two weeks rare Ki-67 nuclear labeling is seen in both groups consistent with a gradual decline in proliferation after the first week of arterial repair in this model; ×400. (C, F) Of note, at four weeks a very unusual and strong late rise in cell proliferation is seen exclusively in the podocan^{-/-} group affecting both alpha-actin positive intimal SMC as well as alpha-actin negative adventitial cells; ×400. FIG. 5G is a bar graph showing the comparison of arterial wall cell proliferation as assessed by Ki-67 expression in podocan WT and podocan^{-/-} groups at one, two and four weeks after arterial injury: Cellular expression in % (independent sample t-test).

FIG. 6 A-F. FIG. 6 shows intimal SMC proliferation during arterial response to injury at two (A, D) and four (B, E) weeks in podocan^{-/-} mice comparing Ki-67 (A-C) and BRDU labeling (D-F) and using BM cells as positive controls (C, F): (A, D) At two week only few distinct intimal proliferative signals are found at a comparable level with Ki-67 and BRDU labeling; ×1000. (B, E) At four weeks, however, increased nuclear labeling with both Ki-67 and BRDU was found in intimal SMC; ×1000. (C, F) Of note, Ki-67 and BRDU labeling was also seen in BM cells serving as positive controls; ×1000.

FIG. 7 A-E. FIG. 7 illustrates a comparison of outgrowth of SMC in aortic explant culture from WT and podocan^{-/-} animals at three days: (A) Light microscopic image of the edge of WT aortic explant shows no cellular outgrowth at three days; ×400; (B) In contrast, at the edge of podocan^{-/-} aortic explants, numerous outgrowing SMC are seen at the same time point reflecting an increase in SMC migratory and possibly also proliferative activity; ×400. At the three day time point, 5 of 8 podocan^{-/-} aortic explants showed similar outgrowth whereas with WT animals 0 of 8 showed such outgrowth. FIG. 7C is a bar graph showing a comparison of WT and podocan^{-/-} SMC migratory activity at low and high serum conditions as assessed by spectrophotometric detection of the number of transmigrated cells: absorption at 588 nm (independent sample t-test). FIG. 7D is a bar graph showing a comparison of WT and podocan^{-/-} SMC proliferative activity at 1% FBS, 10% FBS, and with 10 ng/ml PDGF as assessed by spectrophotometric detection in the MTS assay: absorption at 480 nm (independent sample t-test). FIG. 7E is a bar graph showing a comparison of WT and podocan−/− SMC proliferative activity at 10% FBS, and with 10 ng/ml PDGF in cells transfected with eGFP and podocan as assessed by spectrophotometric detection in the MTS assay: absorption at 480 nm (independent sample t-test).

### DETAILED DESCRIPTION

The instant application demonstrates for the first time that podocan, a novel member of the SLRP family, is a key regulator of arterial response to injury. The podocan−/− genotype was associated with an excessive and prolonged arterial repair process with enhanced SMC activation in vivo as well as in vitro. The delivery of podocan to arterial lesions, whether it be local delivery via a stent or systemic administration that is ultimately localized through podocan's ability to localize to sites of injury by its binding to collagen exposed in the lumen of the vasculature results in modification of SMC proliferation and migration, resulting in the treatment of intimal smooth muscle cell hyperplasia, restenosis following percutaneous coronary intervention, pulmonary hypertension and post-transplant vasculopathy. Likewise, the administration of podocan inhibitors, whether it be local delivery via a stent or systemic administration that is ultimately localized through podocan's ability to localize to sites of injury by its binding to collagen exposed in the lumen of the endothelially damaged/denuded vasculature, results in modification of SMC proliferation. This modification of SMC proliferation can be used to treat vulnerable plaques. Importantly, in contrast to the apoptotic treatments currently used to treat plaques, podocan does not induce SMC apoptosis completely inhibiting SMC function. Rather, podocan normalizes excessive SMC activation (migration and proliferation) in a more physiologic fashion without damaging this critical cell population.

In a further embodiment, the invention provides a method of decreasing or preventing occlusion of a body vessel by smooth muscle cells, comprising administering an agent that promotes podocan signaling or podocan expression. The agent can be a polypeptide, a small molecule, an antibody, an RNAi molecule or any other molecule that promotes podocan signaling or expression.

### DEFINITIONS

As used herein, the term podocan peptide is used to refer to any peptide of the invention comprising the sequence of human podocan peptide as set forth in GenBank accession number NP 714914.2 or AAH30608.1.

(MEGARARGAQLRLGERVRPVGRRSAPGRSRFHQPWRPGASDSAP PAGTMAQSRVLLLLLLLPPQLHLGPVLAVRAPGFGRSGGHSLSPEENEFAEEEPVLVLSPE EPGPGPAAVSCPRDCACSQEGVVDCGGIDLREFPGDLPEHTNHLSLQNNQLEKIYPEELS RLHRLETLNLQNNRLTSRGLPEKAFEHLTNLNYLYLANNKLTLAPRFLPNALISVDFAAN YLTKIYGLTFGQKPNLRSVYLHNNKLADAGLPDNMFNGSSNVEVLILSSNFLRHVPKHLP PALYKLHLKNNKLEKIPPGAFSELSSLRELYLQNNYLTDEGLDNETFWKLSSLEYLDLSS NNLSRVPAGLPRSLVLLHLEKNAIRSVDANVLTPIRSLEYLLLHSNQLREQGIHPLAFQGL KRLHTVHLYNNALERVPSGLPRRVRTLMILHNQITGIGREDFATTYFLEELNLSYNRITSP QVHRDAFRKLRLLRSLDLSGNRLHTLPPGLPRNVHVLKVKRNELAALARGALAGMAQL RELYLTSNRLRSRALGPRAWVDLAHLQLLDIAGNQLTEIPEGLPESLEYLYLQNNKISAVP ANAFDSTPNLKGIFLRFNKLAVGSVVDSAFRRLKHLQVLDIEGNLEFGDISKDRGRLGKE KEEEEEEEEEEEETR) (SEQ ID No. 1) or any functional equivalent, analog or derivative thereof. The podocan protein, functional equivalent, analog or derivative thereof can be encoded by any nucleic acid, such as, the nucleic acid set forth in GenBank accession number NM153703.3 or BC030608 (tggacttgaa tggaaggagc ccgagcccgc ggagcgcagc tgagactggg ggagcgcgtt cggcctgtgg ggcgccgctc ggcgccgggg cgcagcaggt tccatcagcc ctggcgccca ggcgcatctg actcggcacc ccctgcaggc accatggccc agagccgggt gctgctgctc ctgctgctgc tgccgccaca gctgcacctg ggacctgtgc ttgccgtgag ggccccagga tttggccgaa gtggcggcca cagcctgagc cccgaagaga acgaatttgc ggaggaggag ccggtgctgg tactgagccc tgaggagccc gggcctggcc cagccgcggt cagctgcccc cgagactgtg cctgttccca ggagggcgtc gtggactgtg gcggtattga cctgcgtgag ttcccggggg acctgcctga gcacaccaac cacctatctc tgcagaacaa ccagctggaa aagatctacc ctgaggagct ctcccggctg caccggctgg agacactgaa cctgcaaaac aaccgcctga cttcccgagg gctcccagag aaggcgtttg agcatctgac caacctcaat tacctgtact tggccaataa caagctgacc ttggcacccc gcttcctgcc aaacgccctg atcagtgtgg actttgctgc caactatctc accaagatct atgggctcac ctttggccag aagccaaact tgaggtctgt gtacctgcac aacaacaagc tggcagacgc cgggctgccg gacaacatgt tcaacggctc cagcaacgtc gaggtcctca tcctgtccag caacttcctg cgccacgtgc ccaagcacct gccgcctgcc ctgtacaagc tgcacctcaa gaacaacaag ctggagaaga tccccccggg ggccttcagc gagctgagca gcctgcgcga gctatacctg cagaacaact acctgactga cgagggcctg gacaacgaga ccttctggaa gctctccagc ctggagtacc tggatctgtc cagcaacaac ctgtctcggg tcccagctgg gctgccgcgc agcctggtgc tgctgcactt ggagaagaac gccatccgga gcgtggacgc gaatgtgctg acccccatcc gcagcctgga gtacctgctg ctgcacagca accagctgcg ggagcagggc atccacccac tggccttcca gggcctcaag cggttgcaca cggtgcacct gtacaacaac gcgctggagc gcgtgcccag tggcctgcct cgccgcgtgc gcaccctcat gatcctgcac aaccagatca caggcattgg ccgcgaagac tttgccacca cctacttcct ggaggagctc aacctcagct acaaccgcat caccagccca caggtgcacc gcgacgcctt ccgcaagctg cgcctgctgc gctcgctgga cctgtcgggc aaccggctgc acacgctgcc acctgggctg cctcgaaatg tccatgtgct gaaggtcaag cgcaatgagc tggctgcctt ggcacgaggg gcgctggcgg gcatggctca gctgcgtgag ctgtacctca ccagcaaccg actgcgcagc cgagccctgg gcccccgtgc ctgggtggac ctcgcccatc tgcagctgct ggacatcgcc gggaatcagc tcacagagat ccccgagggg ctccccgagt cacttgagta cctgtacctg cagaacaaca agattagtgc ggtgcccgcc aatgccttcg actccacgcc caacctcaag gggatctttc tcaggtttaa caagctggct gtgggctccg tggtggacag tgccttccgg aggctgaagc acctgcaggt cttggacatt gaaggcaact tagagtttgg tgacatttcc aaggaccgtg gccgcttggg gaaggaaaag gaggaggagg aagaggagga ggaggaggaa gaggaaacaa gatagtgaca aggtgatgca gatgtgacct aggatgatgg accgccggac tcttttctgc agcacacgcc tgtgtgctgt gagcccccca ctctgccgtg ctcacacaga cacacccagc tgcacacatg aggcatccca catgacacgg gctgacacag tctcatatcc ccaccccttc ccacggcgtg tcccacggcc agacacatgc acacacatca caccctcaaa cacccagctc agccacacac aactaccctc caaaccacca cagtctctgt cacaccccca ctaccgctgc cacgccctct gaatcatgca gggaagggtc tgcccctgcc ctggcacgca caggcaccca ttccctcccc ctgctgacat gtgtatgcgt atgcatacac accacacaca cacacatgca caagtcatgt gcgaacagcc ctccaaagcc tatgccacag acagctcttg ccccagccag aatcagccat agcagctcgc cgtctgccct gtccatctgt ccgtccgttc cctggagaag acacaagggt atccatgctc tgtggccagg tgcctgccac cctctggaac tcacaaaagc tggcttttat tcctttccca tcctatgggg acaggagcct tcaggactgc tggcctggcc tggcccaccc tgctcctcca ggtgctgggc agtcactctg ctaagagtcc ctccctgcca cgccctggca ggacacaggc acttttccaa tgggcaagcc cagtggaggc aggatgggag agccccctgg gtgctgctgg ggccttgggg caggagtgaa gcagaggtga tggggctggg ctgagccagg gaggaaggac ccagctgcac ctaggagaca cctttgttct tcaggcctgt gggggaagtt ccgggtgcct ttatttttta ttcttttcta aggaaaaaaa tgataaaaat ctcaaagctg atttttcttg ttatagaaaa actaatataa aagcattatc cccaaaaaaa aaaaaaaaaa) (SEQ ID No. 2)

The essential features of the podocan protein is having the conservative consensus of SLRP (L* L** N* * L/I) (SEQ ID No. 3) and a unique pattern of glycosylation and cysteine-rich clusters, apart from several other distinguishing features (Ross et al., 2003; McEwan et al., 2006). Examples of amino acids conserved across the SLRP family of proteins, including podocan, are demonstrated in McEwan et al., Structural Correlations in the Family of Small Leucine-Rich Repeat Proteins and Proteoglycans; Journal of Structural Biology, Vol. 155 (2006), 294-305. Further structure information about human and mouse podocan is set forth in Ross et al., Podocan, a Novel Small Leucine-rich Repeat Protein Expressed in the Sclerotic Glomerular Lesion of Experimental HIV-associated Nephropathy; Journal of Biological Chemistry (2003), Vol. 278, No. 35, 33248-33255. The three dimensional structures of SLRP proteins are demonstrated in Iozzo R V. The biology of the small leucine-rich proteoglycans. Functional network of interactive proteins. J Biol. Chem. 1999; 274:18843-6 and Scott P G, McEwan P A, Dodd C M, Bergmann E M, Bishop P N, Bella J. Crystal structure of the dimeric protein core of decorin, the archetypal small leucine-rich repeat proteoglycan. Proc Natl Acad Sci USA. 2004 Nov. 2; 101(44):15633-8.

The 3-dimensional structure of the podocan protein was initially analyzed using the 3D-PSSM server (available at: http://www.sbg.bio.ic.ac.uk/˜3dpssm/) by submitting the protein sequence directly (Kelley et al, 2000). A leucine rich repeat fold was found at the N-terminal and C-terminal region of podocan. The 3-dimensional structure modeling was next carried out using the PLOP program (kindly provided by Dr Richard A. Friesner, Department of Chemistry and Center for Biomolecular Simulation, Columbia University, New York; Eyrich et al, 1999). The 3-dimensional model of podocan was further analyzed by comparing it with the available crystallographic structures of Decorin (Scott et al, 2004) using the ProSup server (available at: http://lore.came.sbg.ac.at:8080/CAME/CAME_EXTERN/PROSUP/index_html; Lackner et al, 2000). The root mean square deviation (RMSD) of structurally equivalent residues was also calculated, which is a common numerical measure of the difference between 2 protein structures.

As used herein, the term “amino acid” is used to refer to any molecule containing an amine and a carboxylic acid. In one embodiment, the amino acid is attached via a peptide bond.

As used herein in connection with the peptides of the invention, the terms “peptide derivatives” and “peptide analogs” are used interchangeably to refer to peptides in which one or more amino acid residues have been substituted or modified in order to preserve or improve the function of podocan.

As used herein, the term “isolated” means that the material being referred to has been removed from the environment in which it is naturally found, and is characterized to a sufficient degree to establish that it is present in a particular sample. Such characterization can be achieved by any standard technique, such as, e.g., sequencing, hybridization, immunoassay, functional assay, expression, size determination, or the like. Thus, a biological material can be “isolated” if it is free of cellular components, i.e., components of the cells in which the material is found or produced in nature. A protein or peptide that is associated with other proteins and/or nucleic acids with which it is associated in an intact cell, or with cellular membranes if it is a membrane-associated protein, is considered isolated if it has otherwise been removed from the environment in which it is naturally found and is characterized to a sufficient degree to establish that it is present in a particular sample. A protein or peptide expressed from a recombinant vector in a host cell, particularly in a cell in which the protein is not naturally expressed, is also regarded as isolated.

An isolated organelle, cell, or tissue is one that has been removed from the anatomical site (cell, tissue or organism) in which it is found in the source organism. An isolated material may or may not be “purified”. The term “purified” as used herein refers to a material (e.g., a nucleic acid molecule or a protein) that has been isolated under conditions that detectably reduce or eliminate the presence of other contaminating materials. Contaminants may or may not include native materials from which the purified material has been obtained. A purified material preferably contains less than about 90%, less than about 75%, less than about 50%, less than about 25%, less than about 10%, less than about 5%, or less than about 2% by weight of other components with which it was originally associated.

Methods for purification are well-known in the art. For example, polypeptides can be purified by various methods including, without limitation, preparative disc-gel electrophoresis, isoelectric focusing, HPLC, reverse-phase HPLC, gel filtration, affinity chromatography, ion exchange and partition chromatography, precipitation and salting-out chromatography, extraction, and counter-current distribution. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting (FACS)). Other purification methods are possible.

The practice of the present invention will employ, unless indicated specifically to the contrary, conventional methods of molecular biology, cell biology and protein chemistry within the skill of the art, many of which are described below for the purpose of illustration. Such techniques are explained fully in the literature. See, e.g., Sambrook, et al., “Molecular Cloning: A Laboratory Manual” (2nd Edition, 1989); “DNA Cloning: A Practical Approach, vol. I & II” (D. Glover, ed.); “Oligonucleotide Synthesis” (N. Gait, ed., 1984); “Nucleic Acid Hybridization” (B. Hames & S. Higgins, eds., 1985); Perbal, “A Practical Guide to Molecular Cloning” (1984); Ausubel et al., “Current protocols in Molecular Biology” (New York, John Wiley and Sons, 1987); and Bonifacino et al., “Current Protocols in Cell Biology” (New York, John Wiley & Sons, 1999).

The term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

In the context of the present invention insofar as it relates to any of the disease conditions recited herein, the terms “treat”, “treatment”, and the like mean to prevent or relieve or alleviate at least one symptom associated with such condition, or to slow or reverse the progression of such condition. For example, within the meaning of the present invention, the term “treat” also denotes to arrest, delay the onset (i.e., the period prior to clinical manifestation of a disease) and/or reduce the risk of developing or worsening a disease. The term “protect” is used herein to mean prevent, delay or treat, or all, as appropriate, development or continuance or aggravation of a disease in a subject. Within the meaning of the present invention, diseases or conditions include without limitation, restenosis following percutaneous coronary intervention, graft or post-transplant vasculopathy, arterial lesion formation in pulmonary hypertension, cancer and related diseases.

As used herein the term “therapeutically effective” applied to dose or amount refers to that quantity of a compound or pharmaceutical composition that is sufficient to result in a desired activity upon administration to an animal in need thereof. Within the context of the present invention, the term “therapeutically effective” refers to that quantity of a compound or pharmaceutical composition that is sufficient to reduce or eliminate at least one symptom of smooth muscle cell hyperplasia selected from the group consisting of symptoms of restenosis: angina pectoris/chest pain, decreased exercise tolerance and increased shortness of breath; Pulmonary hypertension: shortness of breath, decreased exercise tolerance, signs of R-heart failure (lower extremity swelling, swelling of abdomen), transplant vasculopathy: chest pain, shortness of breath, left and right sided heart failure (leg swelling, abdominal swelling). Methods for detecting these symptoms of smooth muscle cell hyperplasia are well known in the art.

The phrase “pharmaceutically acceptable”, as used in connection with compositions of the invention, refers to molecular entities and other ingredients of such compositions that are physiologically tolerable and do not typically produce untoward reactions when administered to an animal such as a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The terms “administering” or “administration” are intended to encompass all means for directly and indirectly delivering a compound to its intended site of action. The compounds of the present invention can be administered locally to the affected site (e.g., by direct injection into the affected tissue) or systemically. The term “systemic” as used herein includes parenteral, topical, oral, spray inhalation, rectal, nasal, and buccal administration Parenteral administration includes subcutaneous, intravenous, intramuscular, intra-articular, intra-synovial, intrasternal, intrathecal, intrahepatic, intralesional, and intracranial administration. Preferably, administration is parenteral or chronic slow release application (pellet, patch). Even more preferably, administration is local intra-arterial delivery via catheter, balloon, or stent.

The term “animal” means any animal, including mammals and, in particular, humans.

As used in this specification and the appended claims, the singular forms “a,” “an,” and “the” include plural references unless the context clearly dictates otherwise.

The peptides of the invention may be prepared by classical methods known in the art. These standard methods include exclusive solid phase synthesis, automated solid phase synthesis, partial solid phase synthesis methods, fragment condensation, classical solution synthesis, and recombinant DNA technology (See, e.g., Merrifield J. Am. Chem. Soc. 1963 85:2149 and Merrifield et al., 1982, Biochemistry, 21:502).

The terms “polynucleotide” or “nucleotide sequence” mean a series of nucleotide bases (also called “nucleotides”) in DNA and RNA, and mean any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide. This includes single- and double-stranded molecules, i.e., DNA-DNA, DNA-RNA and RNA-RNA hybrids.

The polynucleotides herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

The terms “express” and “expression” mean allowing or causing the information in a gene or DNA sequence to become manifest, for example, producing an non-coding (untranslated) RNA or a protein by activating the cellular functions involved in transcription and translation of a corresponding gene or DNA sequence. A DNA sequence is expressed in or by a cell to form an “expression product” such as RNA or a protein. The expression product itself, e.g. the resulting RNA or protein, may also be said to be “expressed” by the cell.

The terms “vector”, “cloning vector” and “expression vector” mean the vehicle by which a DNA or RNA sequence can be introduced into a host cell, so as to transform the host and clone the vector or promote expression of the introduced sequence. Vectors include plasmids, cosmids, phages, viruses, etc. Vectors may further comprise selectable markers.

The term “host cell” means any cell of any organism that is selected, modified, transformed, grown, used or manipulated in any way, for the production of a substance by the cell, for example, the expression by the cell of a gene, a DNA or RNA sequence, a protein or an enzyme. Host cells can further be used for screening or other assays, as described infra.

As used herein, the term “gene” means a DNA sequence that codes for a particular non-coding (untranslated) RNA or a sequence of amino acids, which comprise all or part of one or more proteins or enzymes, and may include regulatory (non-transcribed) DNA sequences, such as promoter sequences, which determine for example the conditions under which the gene is expressed.

The term “antisense” nucleic acid molecule or oligonucleotide is used in the present disclosure to refer to a single stranded (ss) nucleic acid molecule, which may be DNA, RNA, a DNA-RNA chimera, or a derivative thereof, which, upon hybridizing under physiological conditions with complementary bases in an RNA or DNA molecule of interest, inhibits or activates (in the case of “activating antisense oligonucleotides”) the expression of the corresponding gene by modulating, e.g., RNA transcription, RNA processing, RNA transport, mRNA translation, or RNA stability. As presently used, “antisense” broadly includes RNA-RNA interactions, RNA-DNA interactions, and RNase-H mediated arrest. Antisense nucleic acid molecules can be encoded by a recombinant gene for expression in a cell (see, e.g., U.S. Pat. Nos. 5,814,500 and 5,811,234), or alternatively they can be prepared synthetically (see, e.g., U.S. Pat. No. 5,780,607).

The term “RNA interference” or “RNAi” refers to the ability of double stranded RNA (dsRNA) to suppress the expression of a specific gene of interest in a homology-dependent manner. It is currently believed that RNA interference acts post-transcriptionally by targeting RNA molecules for degradation. RNA interference commonly involves the use of dsRNAs that are greater than 500 bp; however, it can also be mediated through small interfering RNAs (siRNAs) or small hairpin RNAs (shRNAs), which can be 10 or more nucleotides in length and are typically 18 or more nucleotides in length. For reviews, see Bosner and Labouesse, Nature Cell Biol. 2000; 2:E31-E36 and Sharp and Zamore, Science 2000; 287:2431-2433.

As used herein, the term “triplex-forming oligonucleotide” or “triple helix forming oligonucleotide” or “TFO” refers to molecules that bind in the major groove of duplex DNA and by so doing produce triplex structures. TFOs bind to the purine-rich strand of the duplex through Hoogsteen or reverse Hoogsteen hydrogen bonding. They exist in two sequence motifs, either pyrimidine or purine. According to the present invention, TFOs can be employed as an alternative to antisense oligonucleotides and can be both inhibitory and stimulatory. TFOs have also been shown to produce mutagenic events, even in the absence of tethered mutagens. TFOs can increase rates of recombination between homologous sequences in close proximity. TFOs of the present invention may be conjugated to active molecules. For review, see Casey and Glazer, Prog. Nucleic Acid. Res. Mol. Biol. 2001; 67:163-92.

The term “ribozyme” is used herein to refer to a catalytic RNA molecule capable of mediating catalytic reactions on (e.g., cleaving) RNA substrates. Ribozyme specificity is dependent on complementary RNA-RNA interactions (for a review, see Cech and Bass, Annu. Rev. Biochem. 1986; 55:599-629). Two types of ribozymes, hammerhead and hairpin, have been described. Each has a structurally distinct catalytic center. The present invention contemplates the use of ribozymes designed on the basis of the podocan-encoding nucleic acid molecules of the invention to induce catalytic reaction (e.g., cleavage) of podocan, thereby modulating (e.g., inhibiting) a function or expression of podocan. Ribozyme technology is described further in Intracellular Ribozyme Applications: Principals and Protocols, Rossi and Couture ed., Horizon Scientific Press, 1999.

The term “nucleic acid hybridization” refers to anti-parallel hydrogen bonding between two single-stranded nucleic acids, in which A pairs with T (or U if an RNA nucleic acid) and C pairs with G. Nucleic acid molecules are “hybridizable” to each other when at least one strand of one nucleic acid molecule can form hydrogen bonds with the complementary bases of another nucleic acid molecule under defined stringency conditions. Stringency of hybridization is determined, e.g., by (i) the temperature at which hybridization and/or washing is performed, and (ii) the ionic strength and (iii) concentration of denaturants such as formamide of the hybridization and washing solutions, as well as other parameters. Hybridization requires that the two strands contain substantially complementary sequences. Depending on the stringency of hybridization, however, some degree of mismatches may be tolerated. Under “low stringency” conditions, a greater percentage of mismatches are tolerable (i.e., will not prevent formation of an anti-parallel hybrid). See Molecular Biology of the Cell, Alberts et al., 3rd ed., New York and London: Garland Publ., 1994, Ch. 7.

The term “homologous” as used in the art commonly refers to the relationship between nucleic acid molecules or proteins that possess a “common evolutionary origin,” including nucleic acid molecules or proteins within superfamilies (e.g., the immunoglobulin superfamily) and nucleic acid molecules or proteins from different species (Reeck et al., Cell 1987; 50:667). Such nucleic acid molecules or proteins have sequence homology, as reflected by their sequence similarity, whether in terms of substantial percent similarity or the presence of specific residues or motifs at conserved positions.

The terms “percent (%) sequence similarity”, “percent (%) sequence identity”, and the like, generally refer to the degree of identity or correspondence between different nucleotide sequences of nucleic acid molecules or amino acid sequences of proteins that may or may not share a common evolutionary origin (see Reeck et al., supra). Sequence identity can be determined using any of a number of publicly available sequence comparison algorithms, such as BLAST, FASTA, DNA Strider, GCG (Genetics Computer Group, Program Manual for the GCG Package, Version 7, Madison, Wis.), etc.

To determine the percent identity between two amino acid sequences or two nucleic acid molecules, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are, or are about, of the same length. The percent identity between two sequences can be determined using techniques similar to those described below, with or without allowing gaps. In calculating percent sequence identity, typically exact matches are counted.

The determination of percent identity between two sequences can be accomplished using a mathematical algorithm. A non-limiting example of a mathematical algorithm utilized for the comparison of two sequences is the algorithm of Karlin and Altschul, Proc. Natl. Acad. Sci. USA 1990; 87:2264, modified as in Karlin and Altschul, Proc. Natl. Acad. Sci. USA 1993; 90:5873-5877. Such an algorithm is incorporated into the NBLAST and XBLAST programs of Altschul et al., J. Mol. Biol. 1990; 215:403. BLAST nucleotide searches can be performed with the NBLAST program, score=100, word length=12, to obtain nucleotide sequences homologous to sequences of the invention. BLAST protein searches can be performed with the XBLAST program, score=50, word length=3, to obtain amino acid sequences homologous to protein sequences of the invention. To obtain gapped alignments for comparison purposes, Gapped BLAST can be utilized as described in Altschul et al., Nucleic Acids Res. 1997; 25:3389. Alternatively, PSI-Blast can be used to perform an iterated search that detects distant relationship between molecules. See Altschul et al. (1997), supra. When utilizing BLAST, Gapped BLAST, and PSI-Blast programs, the default parameters of the respective programs (e.g., XBLAST and NBLAST) can be used. See ncbi.nlm.nih.gov/BLAST/on the WorldWideWeb. Another non-limiting example of a mathematical algorithm utilized for the comparison of sequences is the algorithm of Myers and Miller, CABIOS 1988; 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

In addition to human podocan, the present invention further provides polynucleotide molecules comprising nucleotide sequences having certain percentage sequence identities to this sequence (such as 85%, 90%, 95% and 99% sequence identity). Such sequences preferably hybridize under conditions of moderate or high stringency as described above, and may include species orthologs.

As used herein, the term “orthologs” refers to genes in different species that apparently evolved from a common ancestral gene by speciation. Normally, orthologs retain the same function through the course of evolution. Identification of orthologs can provide reliable prediction of gene function in newly sequenced genomes. Sequence comparison algorithms that can be used to identify orthologs include without limitation BLAST, FASTA, DNA Strider, and the GCG pileup program. Orthologs often have high sequence similarity. The present invention encompasses all orthologs of podocan.

In a further preferred embodiment the invention provides a method according to the invention, wherein podocan or its functional equivalent is provided with a homing peptide. A homing peptide is any peptide that targets a cell of a selected tissue. Many homing peptides are available in the art. In a preferred embodiment the homing peptides are lung homing peptides, heart homing peptides or tumor homing peptides. Heart homing peptides are for example a CRPPR (SEQ ID No. 4) peptide that at least binds to a Cystein-rich protein 2 receptor, and a CPKTRRVPC (SEQ ID No. 5) peptide that at least binds to a bclO receptor. For further references for heart homing peptides see for instance Zhang, L., Hoffman, J. A., Ruoslahti, E. Molecular profiling of heart endothelial cells. Circulation 112, 1601-1611 (2005). Lung homing peptides are for example Metadherin or GFE-I (CGFECVRQCPERC) (SEQ ID No. 6). For further references for lung homing peptides see for instance Rajotte, D., Ruoslahti, E. Membrane dipeptidase is the receptor for a lung-targeting peptide identified by in vivo phage display. J. Biol. Chem. 274, 11593-11598 (1999); Brown, D., Ruoslahti, E. Metadherin, a novel cell-surface protein in breast tumors that mediates lung metastasis. Cancer cell 5, 365-374 (2004), and U.S. Pat. Nos. 6,933,281; 6,844,324; 6,784,153; 6,610,651; 6,232,287; and 6,174,687.

### Stents

Stents are generally known in the medical arts. As used throughout this specification the terms “stent” and “intraluminal device” are intended to have a broad meaning and encompass any expandable prosthetic device for implantation in a body passageway (e.g., a lumen or artery) to keep a formerly blocked passageway open and/or to provide support to weakened structures (e.g. heart walls, heart valves, venous valves and arteries). The term “stent” and “intraluminal device” has been used interchangeably with terms such as “intraluminal vascular graft” and “expansible prosthesis.”

As used in this specification, the term “body vessel” is intended to have a broad meaning and encompasses any duct (e.g., natural or iatrogenic) within the human body and can include a member selected from the group comprising: artery, vein, common bile duct, pancreatic duct, kidney duct, esophagus, trachea, urethra, bladder, uterus, ovarian duct, fallopian tube, vas deferens, prostatic duct, or lymphatic duct.

Stents are devices which can be delivered percutaneously to treat coronary artery occlusions and to seal dissections or aneurysms of splenic, carotid, iliac and popliteal vessels. Suitable stents useful in the invention are polymeric or metallic. Examples of polymeric stents include stents made with biostable or bioabsorbable polymers such as poly(ethylene terephthalate), polyacetal, poly(lactic acid), and poly(ethylene oxide)/poly(butylene terephthalate) copolymer. Examples of metallic stents include stents made from tantalum or stainless steel. Stents are available in myriad designs; all of which can be used in the present invention and are either commercially available or described in the literature. For example, a self-expanding stent of resilient polymeric material is described in WO 91/12779, entitled “Intraluminal Drug Eluting Prosthesis.” Alternatively, U.S. Pat. No. 4,886,062 describes a deformable metal wire stent. Commercial sources of stents include Johnson & Johnson, Boston Scientific, Cordis, Advanced Catheter Systems, and U.S. Catheter, Inc. PCT publication WO 2004/075,781 describes biodegradable, bioactive polymers that can coat stents and thus release agents such as podocan polypeptide over time in order to heal the artery.

Any DNA encoding podocan protein can be used to coat the stent. Preferably, the DNA sequence of the human cDNA encoding podocan is used. The DNA can be naked or can be incorporated into a vector. Suitable vectors include shuttle vectors, expression vectors, retroviral vectors, adenoviral vectors, adeno-associated vectors and liposomes. See, for example, Walter et al., Circulation 2004; 110; 36-45.

Recombinant genes can be expressed in vivo by implanting the DNA coated stents of the present invention in an artery or vein of a patient. Gene expression is continuous and can optionally be controlled with viral promoters or cell specific promoters.

Methods for coating surfaces are well known in the art and include, for example, spray coating, immersion coating, etc. Any of these methods can be used in the invention (U.S. Pat. No. 6,818,016). For example, a liquid monomeric matrix can be mixed with the DNA and polymerization initiated. The stent can then be added to the polymerizing solution, such that polymer forms over its entire surface. The coated stent is then removed and dried. Multiple application steps can be used to provide improved coating uniformity and improved control over the amount of DNA applied to the stent.

Suitable polymerizable matrix useful for binding the DNA to the stent include any monomeric biocompatible material which can be suspended in water, mixed with DNA and subsequently polymerized to form a biocompatible solid coating. Thrombin polymerized fibrinogen (fibrin) is preferred.

As an equally preferably alternative to a stent coated with podocan-encoding DNA, the stent can be coated with podocan polypeptide. Examples of stents coated with polypeptides can be found, for example, in PCT publication WO 2004/075,781, Swanson N, Hogrefe K, Javed Q, Gershlick A H. In vitro evaluation of vascular endothelial growth factor (VEGF)-eluting stents. Int J. Cardiol. 2003; 92:247-51, U.S. Pat. No. 5,449,382, and Stefanadis et al., Inhibition of plaque neovascularization and intimal hyperplasia by specific targeting vascular endothelial growth factor with bevacizumab-eluting stent: An experimental study, Atheroschlerosis, Mar. 21, 2007.

In another embodiment of the invention, the stents are seeded with genetically modified cells that overexpress podocan (see, for example, Koren et al., Efficient transduction and seeding of human endothelial cells onto metallic stents using bicistronic pseudo-typed retroviral vectors encoding vascular endothelial growth factor, Cardiovascular Revascularization Medicine, Volume 7, Issue 3 (2006), pp. 173-178).

The stent can be placed onto the balloon at a distal end of a balloon catheter and delivered by conventional percutaneous means (e.g. as in an angioplasty procedure) to the site of the restriction or closure to be treated where it can then be expanded into contact with the body lumen by inflating the balloon. The catheter can then be withdrawn, leaving the stent of the present invention in place at the treatment site. The stent may therefore provide both a supporting structure for the lumen at the site of treatment and also a site for instillation of podocan DNA or polypeptide at the lumen wall. The site of instillation can be either an arterial or venous wall.

The stent can be placed in any peripheral or coronary artery or vein. The stent is preferably placed at the site of injury either immediately or soon after mechanical vessel injury.

Stent development has evolved to the point where the vast majority of currently available stents rely on controlled plastic deformation of the entire structure of the stent at the target body passageway so that only sufficient force to maintain the patency of the body passageway is applied during expansion of the stent. Generally, in many of these systems, a stent, in association with a balloon, is delivered to the target area of the body passageway by a catheter system. Once the stent has been properly located (for example, for intravascular implantation the target area of the vessel can be filled with a contrast medium to facilitate visualization during fluoroscopy), the balloon is expanded thereby plastically deforming the entire structure of the stent so that the latter is urged in place against the body passageway. As indicated above, the amount of force applied is at least that necessary to expand the stent (i.e., the applied the force exceeds the minimum force above which the stent material will undergo plastic deformation) while maintaining the patency of the body passageway. At this point, the balloon is deflated and withdrawn within the catheter, and is subsequently removed. Ideally, the stent will remain in place and maintain the target area of the body passageway substantially free of blockage (or narrowing).

See, for example, any of the following patents: U.S. Pat. No. 4,733,665 (Palmaz), U.S. Pat. No. 4,739,762 (Palmaz), U.S. Pat. No. 4,800,882 (Gianturco), U.S. Pat. No. 4,907,336 (Gianturco), U.S. Pat. No. 5,035,706 (Gianturco et al), U.S. Pat. No. 5,037,392 (Hillstead), U.S. Pat. No. 5,041,126 (Gianturco), U.S. Pat. No. 5,102,417 (Palmaz), U.S. Pat. No. 5,147,385 (Beck et al.), U.S. Pat. No. 5,282,824 (Gianturco), U.S. Pat. No. 5,316,023 (Palmaz et al.), Canadian patent 1,239,755 (Wallsten), Canadian patent 1,245,527 (Gianturco et al.), Canadian patent application number 2,134,997 (Perm et al.), Canadian patent application number 2,171,047 (Penn et al.), Canadian patent application number 2,175,722 (Penn et al.), Canadian patent application number 2,185,740 (Penn et al.), Canadian patent application number 2,192,520 (Penn et al.), International patent application PCT/CA97/00151 (Penn et al.), International patent application PCT/C A97/00152 (Penn et al.), and International patent application PCT/CA97/00294 (Penn et al.), for a discussion on previous stent designs and deployment systems.

The administration of stents that carry therapeutic coatings, such as one or more polymeric coatings including pharmacologically active agents, have been utilized to reduce some of the problems created by the implantation of stents, such as restenosis and other biocompatibility responses to the foreign implant. See also WO 2006/009,883 for a discussion of coated stents.

### Methods of Diagnosis

In one embodiment the invention provides a method for diagnosing a condition of vasculature of an individual, comprising obtaining a sample from said individual and measuring a level of podocan polypeptide or mRNA expression in said sample. A condition of vasculature as used herein is a status of health or development of vasculature of an individual, such as a pathological or a physiological status. A sample as used in the invention is for example a sample of a bodily fluid, such as blood or lymph. A sample is alternatively obtained from a bronchoalveolar lavage (BAL), Transbronchial biopsy (TBB), or Endomyocardial heart biopsy or from arteries by directional coronary atherectomy (DCA). In one embodiment a sample is a vascular cellular sample. A vascular cellular sample of the invention is any sample comprising cells that were located adjacent to or part of a vascular tissue.

An expression level of podocan can be measured in alternative ways. An expression level of podocan can be measured from any product of a podocan mRNA. For example the level of podocan protein, or the level of a derivative of podocan protein is measured. An expression level of podocan is for example performed through an immunodetecting technique, such as immunohistochemistry, immunofluorescence or immunoblotting. Alternatively expression levels are determined with a PCR technique, for instance quantitative real time PCR. In the art many other techniques for determining an expression level are available, such as multiple microarray techniques. In a preferred embodiment a method according to the invention is provided, wherein measuring is performed through PCR, a microarray technique, immunohistochemistry, immunofluorescence or immunoblotting.

In one embodiment the invention provides a method for diagnosing a condition of vasculature of an individual, wherein said condition of vasculature of said individual is associated with a disorder in said individual and wherein said disorder is a vascular proliferative disease. Diagnosis is either directed to a local, a regional or a systemic condition of vasculature of an individual. A vascular proliferative disorder is any disease wherein vasculature of an individual proliferates. Proliferation typically refers to cell multiplication, but generally, as in most vascular proliferative disorders, it also involves growth of at least some individual cells. Non-limiting examples of vascular proliferative disorders are: idiopathic pulmonary hypertension, chronic hypoxic pulmonary hypertension, systemic hypertension, artherosclerosis, post-angioplasty restenosis, vasculopathy, diabetic vasculopathy, vascular injury, vasculitis, arteritis, capillaritis or carcinoma. In a preferred embodiment, the invention provides a method for diagnosing a condition of vasculature of an individual, wherein said vascular proliferative disease is selected from the following: pulmonary hypertension, carcinoma or vascular injury.

1. Exemplary Recombinant Expression Systems

### High Yield Expression of Bioactive Recombinant Human Podocan in Insect Cells.

Podocan requires glycosylation for its biological function. Insect cell based baculovirus systems permit production of glycosylated recombinant protein. Additionally, higher yields of recombinant protein expression can be obtained by using baculovirus systems instead of mammalian expression system.

To produce glycosylated recombinant podocan, the cDNA sequence encoding human podocan (GenBank Locus BC030608) is cloned into pIa/Bac 3C/LIC baculovirus vector (Cat# 71731-3, Novagen, Madison, Wis.), then transformed into NovaBlue GigaSingles competent cells with blue/white selection on LB plates containing X-gal and ampicillin. Positive plasmids with the expected insert are analyzed by restriction digestion mapping and then sequenced to ensure the plasmid in mutation-free. The selected plasmid is subsequently transfected into the insect cell line sf9 (Cat#71259-4, Novagen Co.) with GeneJuice Transfection reagent.

After a large-scale culture (>1 L) of transfected Sf9 cells in HyQ SFX-insect culture medium (Cat.# SH30278.02, HyClone Co.), recombinant human podocan is purified from total protein extraction using an InsectDirect™ System-Insect RoboPop™ Ni-NTA His·Bind® Purification Kit (Cat#71257-3, Novagen Co.). The yield of purified recombinant protein is up to 40 mg/1 L transfected cells. To ensure that podocan protein is glycosylated, the purified product is run on a SDS-PAGE gel followed by Coomassie blue staining and GelCode glycoprotein staining (Cat.# 24562, Pierce Co.). Recombinant protein concentration is determined by BCA (bicinchoninic acid) assay.

### High Yield Expression of Recombinant Human Podocan Protein in Cho Cells.

The cDNA sequence encoding human podocan (GenBank Locus BC030608) is cloned into pcDNA™ 4HisMAX (Cat.#V864-20), then transformed into its compatible competent cells with blue/white selection on LB plates containing X-gal and ampicillin. Positive plasmids with human podocan cDNA are analyzed by restriction digestion mapping and then sequenced to ensure the plasmid in mutation-free. The selected plasmid is subsequently transfected into CHO-S cells (Cat# R800-07, Invitrogen Co.) with FreeStyle MAX reagent (Cat# 16447, Invitrogen CO.). To obtain a high efficient transfection, viability of cells need to be >95% before the transfection.

For a large-scale generation of recombinant protein, transfected CHO cells are cultured in GIBCO® FreeStyle™ CHO Expression Medium (Cat.# 12651, Invitrogen Co.) at 37° C., 8% CO₂ on a shaker platform rotating at 135 rpm. Protein expression is detectable within 4-8 hours of transfection, with maximal protein yield between 1-7 days post-transfection. Recombinant human podocan is purified from total protein extraction using ProBond™ Metal-Binding Resin (Cat# R801, Invitrogen Co.). Purified product is run on a SDS-PAGE gel followed by Coomassie blue staining and GelCode glycoprotein staining (Cat.# 24562, Pierce Co.). Recombinant protein concentration is determined by BCA assay.

To further test biological function of the purified glycosylated podocan, its ability to bind and regulate collagen type I assembly by fibrillogenesis assay will be determined. First, collagen type I is dissolved at 1 mg/mL in 10 mM acetic acid overnight at 4° C. Aliquots are mixed on ice with equal volumes of double concentrated fibrillogenesis buffer (50 mM sodium dihydrogenphosphate, 10 mM potassium dihydrogenphosphate and 270 mM NaCl at pH 7.4 to yield 60 mM phosphate with 135 mM NaCl) in 1.0 mL quartz cuvettes (Hellma, Germany). Recombinant glycosylated podocan are added to the reaction solution before the start of fibrillogenesis. As a control, unglycosylated recombinant podocan generated by E. coli system will be tested as well. Fibril formation took place at 37° C. over a period of 1000 min. Optical density will be measured at 313 nm every 3 min. Accordingly, kinetics of the fibrillogenesis will be illustrated by the turbidity curve.

1. Use of the Nucleic Acid Molecules of the Invention to Modulate Podocan Function and Expression

The present invention provides podocan-specific antisense oligonucleotides, RNA interference (RNAi) molecules, ribozymes, and triple helix forming oligonucleotides (TFOs) which can be effectively used to inhibit podocan function. In conjunction with these antisense oligonucleotides, RNA interference (RNAi) molecules, ribozymes, and triple helix forming oligonucleotides (TFOs), the present invention provides a method of inhibiting podocan function in a cell comprising administering said molecules to the cell.

### Methods for the Use of the Compounds of the Invention and Compositions Thereof

The compounds of the invention and compositions thereof are useful in a wide variety of therapeutic applications including, but not limited to, the treatment of tissue damage associated with intimal smooth muscle hyperplasia, restenosis following percutaneous coronary intervention, graft vasculopathy, and pulmonary hypertension

Such methods include administering a composition of this invention to an animal/patient in an amount effective to treat tissue damage.

The optimal therapeutically effective amount of a compound or composition of this invention may be determined experimentally, taking into consideration the exact mode of administration, the form in which the drug is administered, the indication toward which the administration is directed, the subject involved (e.g., body weight, health, age, sex, etc.), and the preference and experience of the physician or veterinarian in charge.

The efficacy of the peptides and compositions of this invention can be determined using the in vitro and in vivo assays described in the Examples section, below.

Following methodologies which are well-established in the art, effective doses and toxicity of the peptides and compositions of the present invention, which performed well in in vitro tests, can be determined in studies using small animal models (e.g., mice, rats or dogs) in which they have been found to be therapeutically effective and in which these drugs can be administered by the same route proposed for the human trials.

For any pharmaceutical composition used in the methods of the invention, dose-response curves derived from animal systems can be used to determine testing doses for administration to humans. In safety determinations for each composition, the dose and frequency of administration should meet or exceed those anticipated for use in any clinical trial.

As disclosed herein, the dose of the compound in the compositions of the present invention is determined to ensure that the dose administered continuously or intermittently will not exceed an amount determined after consideration of the results in test animals and the individual conditions of a patient. A specific dose naturally varies (and is ultimately decided according to the judgment of the practitioner and each patient's circumstances) depending on the dosage procedure, the conditions of a patient or a subject animal such as age, body weight, sex, sensitivity, feed, dosage period, drugs used in combination, seriousness of the disease, etc.

Toxicity and therapeutic efficacy of the compositions of the invention can be determined by standard pharmaceutical procedures in experimental animals, e.g., by determining the LD₅₀ (the dose lethal to 50% of the population) and the ED₅₀ (the dose therapeutically effective in 50% of the population). The dose ratio between therapeutic and toxic effects is the therapeutic index and it can be expressed as the ratio ED₅₀/LD₅₀.

### Delivery of the Peptides of the Invention to the Target Damaged Tissue

All known peptide delivery methods can be used to deliver the peptides of the present invention to the target damaged cells and tissues. The specific type of delivery useful for a given peptide is determined by its specific size, flexibility, conformation, biochemical properties of constituent amino acids, and amino acid arrangement. Peptide composition also determines, in part, the degree of protein binding, enzymatic stability, cellular sequestration, uptake into non-target tissue, clearance rate, and affinity for protein carriers. Other aspects independent of peptide composition must also be considered, such as cerebral blood flow, diet, age, sex, species (for experimental studies), dosing route, and effects of existing pathological conditions.

Examples of delivery methods useful for obtaining effective tissue delivery of the peptides of the invention (and effective passage through the blood-brain-barrier in case of brain tissues), include, without limitation (reviewed, e.g., in Witt and Davis, AAPS Journal, 2006; 8(1): E76-E88.):
(i) invasive procedures (e.g., direct injection by, e.g., using an external pump or i.v. line), transient osmotic opening, shunts, and biodegradable implants);
(ii) pharmacologically-based approaches to increase the tissue delivery by chemical modification of the peptide molecule itself, or by the attachment or encapsulation of the peptide in a substance that increases permeability, stability, bioavailability, and/or receptor affinity; in addition, modification of a peptide structure and/or addition of constituents (e.g., lipophilicity enhancers, polymers, antibodies) may enhance local peptide concentration in the target tissue;
(iii) physiologic-based strategies which exploit various carrier mechanisms; these strategies can be combined, dependent of the nature of a given peptide, creating “hybrid” peptides, resulting in synergistic delivery and end-effect; and
(iv) stents coated with podocan polypeptide.

Oral Delivery. Contemplated for use herein are oral solid dosage forms, which are described generally in Remington's Pharmaceutical Sciences, 18th Ed. 1990 (Mack Publishing Co. Easton Pa. 18042) at Chapter 89, which is herein incorporated by reference. Solid dosage forms include tablets, capsules, pills, troches or lozenges, cachets, pellets, powders, or granules. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given by Marshall, K. In: Modern Pharmaceutics Edited by G. S. Banker and C. T. Rhodes Chapter 10, 1979, herein incorporated by reference. In general, the formulation will include a peptide of the invention (or chemically modified forms thereof) and inert ingredients which allow for protection against the stomach environment, and release of the biologically active material in the intestine.

Also contemplated for use herein are liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

As discussed above, the peptides may be chemically modified so that oral delivery of the derivative is efficacious. Generally, the chemical modification contemplated is the attachment of at least one moiety to the component molecule itself, where said moiety permits (a) increase in peptide stability (e.g., by inhibition of proteolysis) and (b) efficient uptake into the blood stream from the stomach or intestine. As discussed above, common delivery-improving peptide modifications include PEGylation or the addition of moieties such as propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane (see, e.g., Abuchowski and Davis (1981) “Soluble Polymer-Enzyme Adducts,” in Enzymes as Drugs. Hocenberg and Roberts, eds. (Wiley-Interscience: New York, N.Y.) pp. 367-383; and Newmark, et al. (1982) J. Appl. Biochem. 4:185-189).

For oral formulations, the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the peptide (or derivative) or by release of the peptide (or derivative) beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (i.e. powder), for liquid forms a soft gelatin shell may be used. The shell material of cachets could be thick starch or other edible paper. For pills, lozenges, molded tablets or tablet triturates, moist massing techniques can be used.

The peptide (or derivative) can be included in the formulation as fine multiparticulates in the form of granules or pellets of particle size about 1 mm. The formulation of the material for capsule administration could also be as a powder, lightly compressed plugs, or even as tablets. These therapeutics could be prepared by compression.

Colorants and/or flavoring agents may also be included. For example, the peptide (or derivative) may be formulated (such as by liposome or microsphere encapsulation) and then further contained within an edible product, such as a refrigerated beverage containing colorants and flavoring agents.

One may dilute or increase the volume of the peptide (or derivative) with an inert material. These diluents could include carbohydrates, especially mannitol, lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress, and Avicel.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. The disintegrants may also be insoluble cationic exchange resins. Powdered gums may be used as disintegrants and as binders and can include powdered gums such as agar, Karaya or tragacanth. Alginic acid and its sodium salt are also useful as disintegrants.

Binders may be used to hold the peptide (or derivative) agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the peptide (or derivative).

An antifrictional agent may be included in the formulation of the peptide (or derivative) to prevent sticking during the formulation process. Lubricants may be used as a layer between the peptide (or derivative) and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the peptide (or derivative) into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants could be present in the formulation of the protein or derivative either alone or as a mixture in different ratios.

Additives which potentially enhance uptake of the peptide (or derivative) are for instance the fatty acids oleic acid, linoleic acid and linolenic acid.

Controlled release oral formulations may be desirable. The peptide (or derivative) could be incorporated into an inert matrix which permits release by either diffusion or leaching mechanisms, e.g., gums. Slowly degenerating matrices may also be incorporated into the formulation. Some enteric coatings also have a delayed release effect. Another form of a controlled release is by a method based on the Oros therapeutic system (Alza Corp.), i.e. the drug is enclosed in a semipermeable membrane which allows water to enter and push drug out through a single small opening due to osmotic effects.

Other coatings may be used for the formulation. These include a variety of sugars which could be applied in a coating pan. The peptide (or derivative) could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide the optimum film coating. Film coating may be carried out in a pan coater or in a fluidized bed or by compression coating.

Parenteral delivery. Preparations according to this invention for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

Preferably, the L-peptides of the invention (e.g., L-CEFH) (SEQ ID No. 7) are administered to treat diseases related to NO damage by parental i.v. injection in a standard physiological solution. The D-peptides of the invention (e.g., D-CEFH) (SEQ ID No. 8) can be administered using any standard administration technique known in the art, such as oral administration.

It is also possible to deliver podocan via gene therapy techniques. For example, podocan vectors can be introduced in vivo as a naked DNA plasmid. Naked DNA vectors for gene therapy can be introduced into the desired host cells by methods known in the art, e.g., use of a gene gun, or use of a DNA vector transporter. Receptor-mediated DNA delivery approaches can also be used (Curiel et al., Hum. Gene Ther. 1992, 3:147-154; Wu and Wu, J. Biol. Chem. 1987, 262:4429-4432). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir et al., C.P. Acad. Sci. 1988, 321:893; PCT Publication Nos. WO 99/01157; WO 99/01158; WO 99/01175).

In the gene therapy embodiment of the invention, the method comprises administering to a mammal harboring a body vessel occlusion an amount of a vector effective to treat the occlusion, wherein the vector inserts itself into the cells in the area of or adjacent to the occlusion and these cells then express greater than normal levels of podocan. Preferably, the vector is a virus-based vector. As disclosed herein, the preferred virus-based vector for use in the method of the invention is an adenoviral, AAV and retroviral vector (all of which are replication defective). In the gene therapy embodiment of the invention, the gene therapy vector can be delivered via a gene-elutin stent. See for, example, Sharif et al., Human Gene Therapy 17:741-750 (July 2006).

### EXAMPLES

The present invention will be better understood by reference to the following non-limiting examples.

### Example 1

### Podocan Expression in Injured Arterial Wall and in Human Atheroma

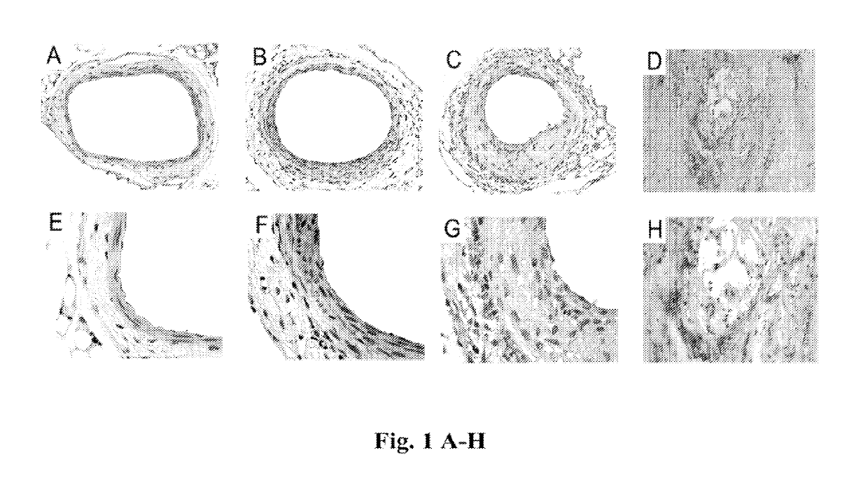

Whether there was an association between podocan protein expression and arterial repair was tested using immunohistochemical staining for podocan in mouse femoral artery and human atheroma. Podocan staining was absent in non-injured femoral arteries of wild-type (WT) mice as shown in FIGS. 1A and 1E. In contrast, expression of podocan was detected in injured femoral arteries of WT mice at four weeks after arterial injury. At this time point, podocan expression was seen in the intracellular space of SMC as well as extracellular deposits of podocan around medial and neointimal SMC were seen (FIGS. 1B and 1F). In injured femoral artery of podocan-deficient mice, however, podocan immuno-labeling was completely absent, confirming the lack of podocan expression in the abundant SMC in the enlarged neointima of podocan^{-/-} mice (FIGS. 1C and 1G). When using an anti-human podocan antibody on sections of human carotid atheroma, strong podocan expression was found in areas of plaque repair marked by neovascularization and cellular infiltrates in the extracellular space (FIGS. 1D and 1H).

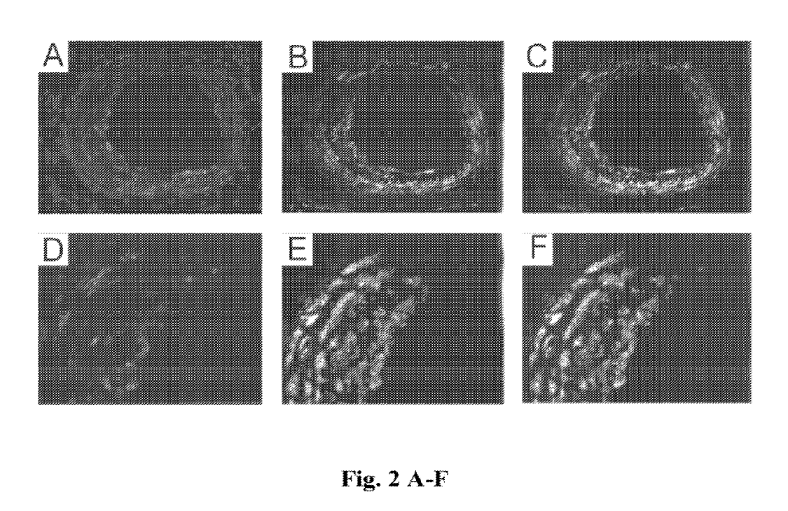

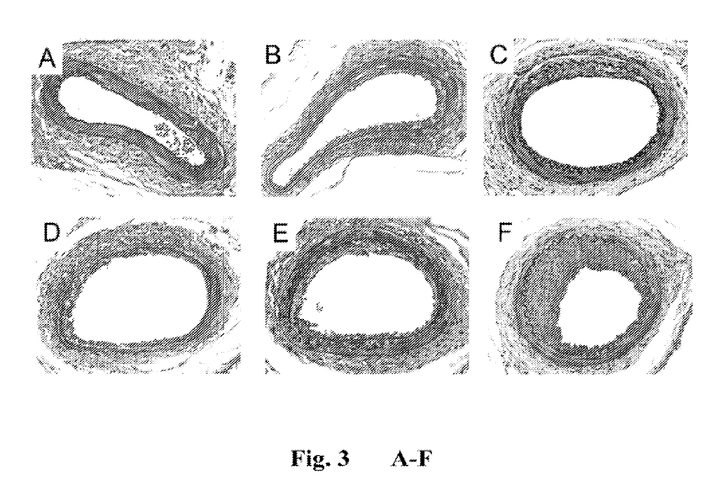

Immunofluorescent double-labeling for smooth muscle alpha-actin and podocan in injured femoral artery of WT mice at two weeks after arterial injury showed medial and nascent neointimal SMC alpha-actin expression by red fluorescent (Texas-Red) signals with a predominance in luminal differentiating SMC. (FIGS. 2A and 2D). Of note, green fluorescent signals (FITC) indicating the strong presence of podocan was seen in the cytoplasm of a majority of medial and neointimal cells but was completely absent in adventitial cells (FIGS. 2B and 2E). Overlay of Texas-Red and FITC signals indicates that podocan expression preceded alpha-actin expression in not yet fully differentiated neointimal SMC at two weeks (FIGS. 2C and 2F).

### Discussion

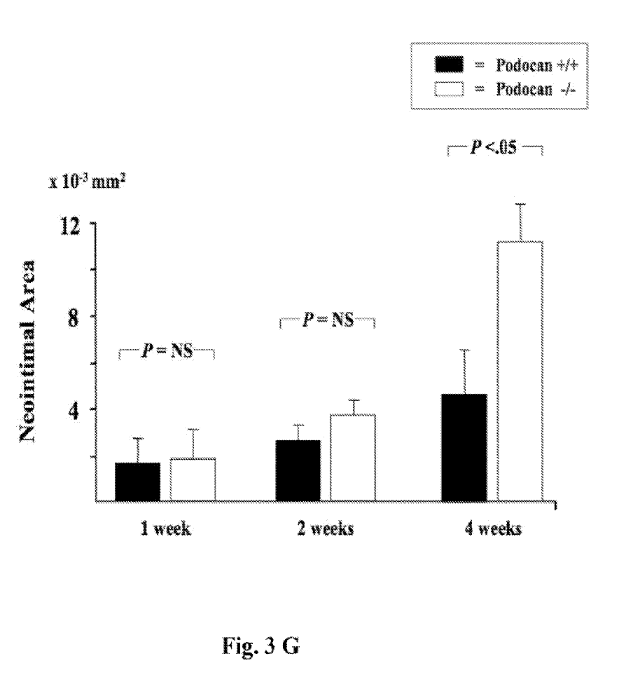

Arterial lesion formation involves migration of activated SMC from the media to the intimal space and subsequent SMC proliferation and ECM synthesis^{3,19-21}. Podocan appears to be an important component of the local regulatory control for arterial injury repair for several reasons. Podocan was detectable at the site of vascular injury and ensuing SMC activation. Selective expression of podocan protein was observed in SMC in the medial and neointimal compartment at four weeks after arterial injury in WT mice (FIG. 3). Podocan was found in intra- as well as extracellular location in a significant number of cells. In contrast, podocan protein expression as detected by immunostaining was completely absent in the media of non-injured WT arteries indicating a selective podocan protein synthesis by SMC after arterial injury (FIG. 3). In hyperplastic neointima of podocan^{-/-} mice, podocan protein was completely absent. Together, these findings demonstrate that in the murine model, podocan serves to limit the proliferative repair response to vascular injury.

To explore whether these findings are present in humans as well, human atheroma were examined for the presence of podocan protein (FIGS. 1 and 2). Although human atheromas differ from the murine model in that they contain a significant amount of inflammatory cells/macrophages they still contain a significant amount of SMCs in the fibrous cap and in areas of plaque repair, especially in carotid lesions^{1,22,23}. Using an antibody raised against human podocan, podocan antigen was detected in SMC-rich areas with higher cell density and in the vicinity of intra-plaque neovascularization (FIGS. 1 and 2).

### Method: Generation of Podocan Deficient Mice

A podocan targeting vector was constructed by inserting a neomycin cassette into podocan wild-type genomic sequence, which was subsequently incorporated into the mouse genome by recombination. This insertion led to the targeted deletion of exons III through VIII of the podocan gene, consequently abolishing podocan expression. After ES cell transfection, selection of positive ES cells and blastocyst injection, the resulting chimeric males were crossed with C57/BL6 female mice. Subsequent heterozygous agouti offspring were bred to homozygosity. The genotyping of resulting mice was performed using RT-PCR and Southern blotting. Mice were housed at the Center for Laboratory Animal Sciences at The Mount Sinai Medical Center, New York. Mice received standard rodent chow (Mouse diet # 5015, PMI Nutrition International) and tap water ad libitum. Procedures and animal care were approved by the Institutional Animal Care and Use Committee, and were in accordance with the “Guide for the Care and Use of Laboratory Animals” (National Research Council. Washington, D.C.: National Academy Press 1996).

### Method: Endothelial Denudation Injury of Mouse Femoral Artery

Mice of podocan^{-/-} and WT genotype (podocan^{+/+}) underwent femoral arterial injury (n=45). Mice were anesthetized with intra-peritoneal pentobarbital sodium (40 mg/kg) (Nembutal®, Abbott Laboratories). Removal of the endothelium of the common femoral artery using a surgical microscope was achieved by 3 passages of a 0.25 mm angioplasty guide wire (Advanced Cardiovascular Systems). The protocol, as well as the degree of injury applied to the vessel wall has been standardized, validated, and described in detail in previous studies^{17,18}.

### Method: Tissue Preparation, Histology and Immunostaining

Animals were sacrificed one, two and four weeks after arterial injury and perfusion-fixed with 4% paraformaldehyde (PFA) in phosphate buffered saline (PBS) at 100 mm Hg for 10 minutes and their hindlimbs excised en bloc. Specimens were fixed overnight in 4% PFA in PBS and decalcified in 10% formic acid. Two 2-mm thick cross sections were cut from each hindlimb at the level of injury in the common femoral artery and processed for paraffin embedding. Sequential sections (4 μm thick) were stained with Masson's Trichrome and hematoxylin-eosine. Immunohistochemical staining was performed with polyclonal rabbit antibodies against murine and human podocan (generated in the Klotman laboratory; 1:45 and 1:25, respectively), von Willebrand Factor (Dako; 1:1000), smooth muscle alpha-actin (Sigma; 1:300), and Ki-67 (R&D Systems; 1:150). Tissue sections were quenched with 3% hydrogen peroxide, blocked with 1% bovine serum albumin in PBS and incubated with the primary antibodies at 37° C. for 2 hours. After washing in PBS, bound primary antibody was detected using an appropriate biotinylated secondary antibody for 15 minutes at 37° C. Sections were washed in PBS, reacted with horseradish peroxidase-conjugated streptavidin, developed with 3,3′-diaminobenzidine and counterstained with hematoxylin. Negative controls were prepared by substitution of the primary antibody with the respective control IgG. Immunofluorescence double labeling was performed using fluorescein isothiocyanate (FITC)- and Texas Red-conjugated secondary antibodies (Jackson Immuno Laboratories) in combination with nuclear DAPI counterstaining.

### Method: Human Specimens

Paraffin blocks of formalin-fixed atherosclerotic plaque tissue were obtained from carotid endarterectomy specimens (n=7) and coronary atherectomy specimens from primary and restenotic target lesions (n=6). The use of excess anonymous clinical tissue was approved by the institutional review board.

### Example 2

### Effect of Podocan Genotype on Arterial Response to Injury and SMC Activation In Vivo

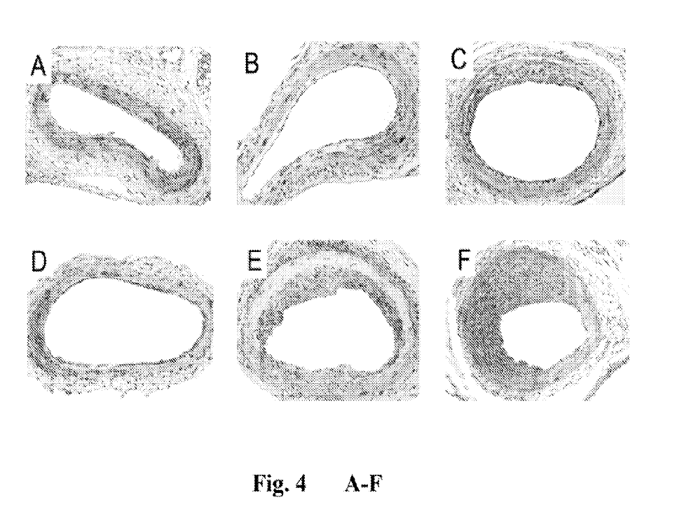

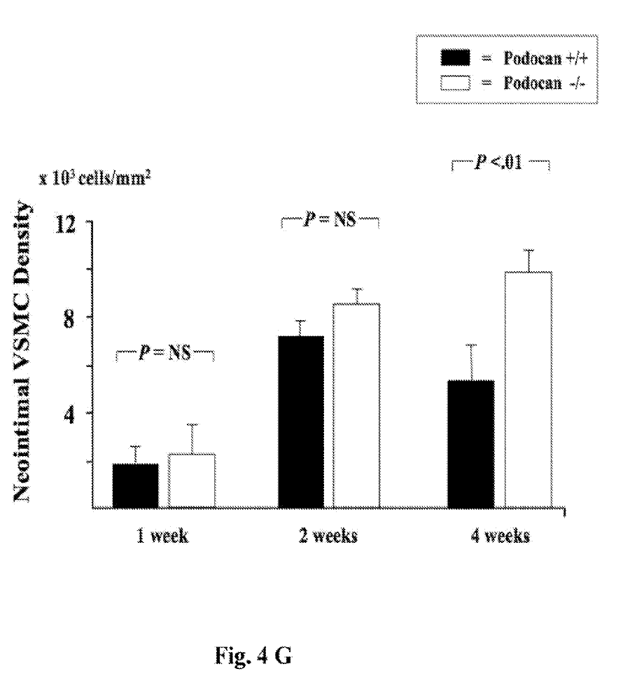

The effect of podocan genotype on the time course of femoral arterial response to injury in WT (n=24) and podocan^{-/-} mice (n=25) is shown in FIG. 3. Neointima formation was measured at one, two, and four weeks after denudating injury. At one and two weeks, no significant difference in neointima area was found when comparing podocan^{-/-} and WT mice (one week: 2.0═0.9 vs. 1.8±0.8×10-³ mm², P=NS; two weeks: 3.8±1.0 vs. 2.9±0.9×10-³ mm², P=NS) (FIG. 3G). At 4 weeks, however, podocan^{-/-} mice showed a strong and significant increase in neointima area compared to WT mice (11.6±1.8 vs. 4.4±1.3×10-³ mm², P<0.05) (FIG. 3G). Media area and total vessel area were not different between the two groups. Consistently, neointima to media ratio was strongly increased in podocan^{-/-} mice at four weeks as well (3.04±0.44 vs. 1.14±0.15; P<0.01). At the earlier one and two week time points neointimal SMC density, as detected by anti-smooth muscle alpha-actin immunostaining (FIG. 4), did not show a statistically significant difference between podocan^{-/-} and WT mice (one week: 2078±978 vs. 1958±934×10³ cells/mm², P=NS; two weeks: 8822±2078 vs. 7823±1934×10³ cells/mm², P=NS) (FIG. 4G). At four weeks, however, concordant with the late increase in neointima area, a significantly increased SMC density was found in the neointima of mice lacking podocan (9989±2978 vs. 5813±2012×10³ cells/mm², P<0.05) (FIG. 4G).

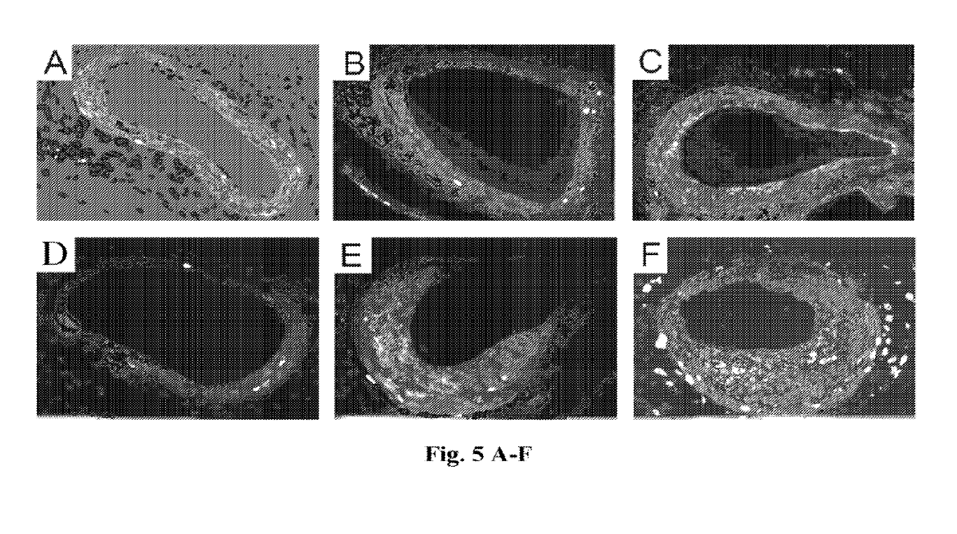

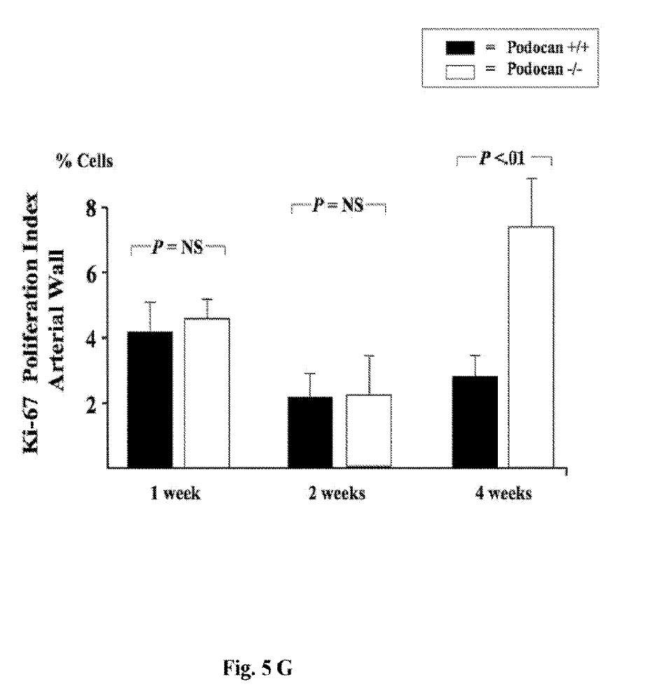

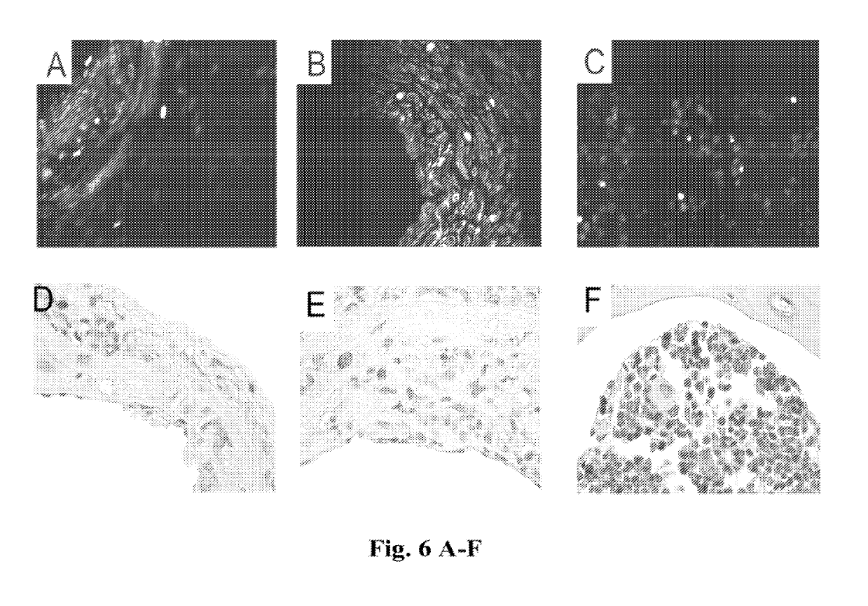

The cell proliferation antigen Ki-67 marker was used to analyze cellular proliferative events in the arterial wall. An unusual pattern of late SMC activation in response to injury repair was seen in podocan^{-/-} mice (FIGS. 5 and 6). At one week, 4.4±1.0% of arterial wall cells expressed Ki-67 in podocan^{-/-} mice and 4.1±0.8% in WT mice (P=NS) (FIG. 5G). At two weeks, Ki-67 expression had decreased similarly in both groups as usually seen in experimental models of arterial injury (2.3±1.1% vs. 2.2±0.9%; P=NS) (FIG. 5G). However, podocan^{-/-} mice lacking the strong medial and neointimal expression of podocan seen in injured femoral arteries of WT animals showed an unusual late and significant increase in Ki-67 expression at four weeks (7.3±1.9% vs. 2.4±1.0%; P<0.05). This finding is consistent with a sustained SMC activation (FIG. 5G) at a time when proliferation has normally ceased. Ki-67 proliferation measurements were corroborated by using anti-BRDU labeling after BRDU injection in animals 4 h prior to sacrifice (FIG. 6).

Morphometric analysis of von Willebrand Factor (vWF) immunostaining along the luminal surface of injured arteries showed no difference in the degree of reendothelialization of the denuded vascular surface in podocan animals compared to WT controls at all time points (one week: 27±2% vs. 29±4%, P=NS; two weeks: 57±5% vs. 54±4%, P=NS; four weeks: 79±4% vs. 84±4%, P=NS). Data not shown.

### Discussion.

When analyzing the time course of arterial response to injury it became evident that the significant increase in neointima formation in the podocan^{-/-} genotype occurs late between two and four weeks after injury (FIG. 3). Interestingly, this is exactly the time when ECM synthesis and remodeling occurs in the neointima, following the initial stage of intimal cell recruitment. This time line is well established in several different models of experimental arterial lesion formation^{21,24-28}. The observation that podocan protein expression was high at this later time in WT mice and that in the absence of podocan (in −/− mice), neointima growth was accelerated suggested an inhibitory effect of podocan on intimal SMC. Podocan therefore was hypothesized to serve as an ECM stop signal limiting arterial repair and preventing exuberant neointima formation.

To further test this hypothesis, the effect of podocan genotype on SMC proliferation in vivo as well as in vitro was analyzed. Consistent with the late increase in neointima formation a significant increase in late nuclear expression of the proliferation marker Ki-67 was observed in the injured arterial wall of podocan^{-/-} mice at 4 weeks (FIGS. 5 and 6). This is especially remarkable given that the natural history of arterial wall cell proliferation in this and other models peaks during the first two weeks and tapers off at later time points^{3,21,28}. Along with the unusual late increase in proliferative signals, an increase in neointimal SMC density was found at four weeks in the podocan^{-/-} mice when compared to the WT genotype (FIG. 4). Normally, by four weeks, SMC density declines due to a decreased rate of SMC proliferation and ongoing ECM synthesis^{29,30}. In the knockout mice, just the opposite happened, with increased proliferation of the neointima. Thus, podocan acts as a negative feedback regulator of SMC activation and arterial repair.

### Method: Computer Assisted Morphometry

Histomorphometric evaluation of arterial response to injury was performed at one, two and four weeks by investigators blinded to the study design. A computer-assisted planimetry system was used (Software: Image Pro Plus 3.0.1). Endothelial cell coverage of the luminal surface was assessed by ×400 microscopic examination of sections with nuclear hematoxylin counterstain and staining for vWF when both an endothelial cell nucleus and immunostaining were present. Neointimal area was assessed by hematoxylin eosin and Masson's trichrome staining. SMC density and arterial wall cell proliferation (Ki-67 labeling) were measured as smooth muscle alpha-actin positive cells per area and as percentage Ki-67 positive cells from total cells with nuclear counterstaining. No significant inter- or intra-observer variations were noted.

### Example 3

### Effects of Podocan Genotype on SMC Activation In Vitro

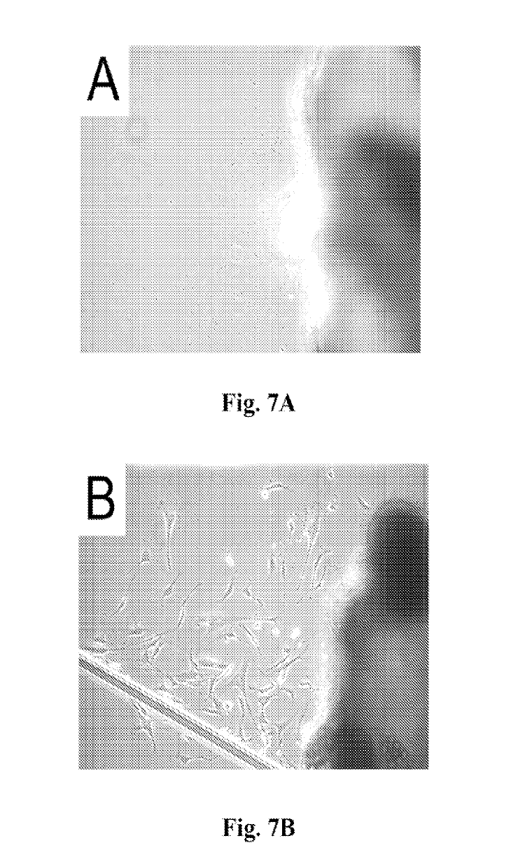

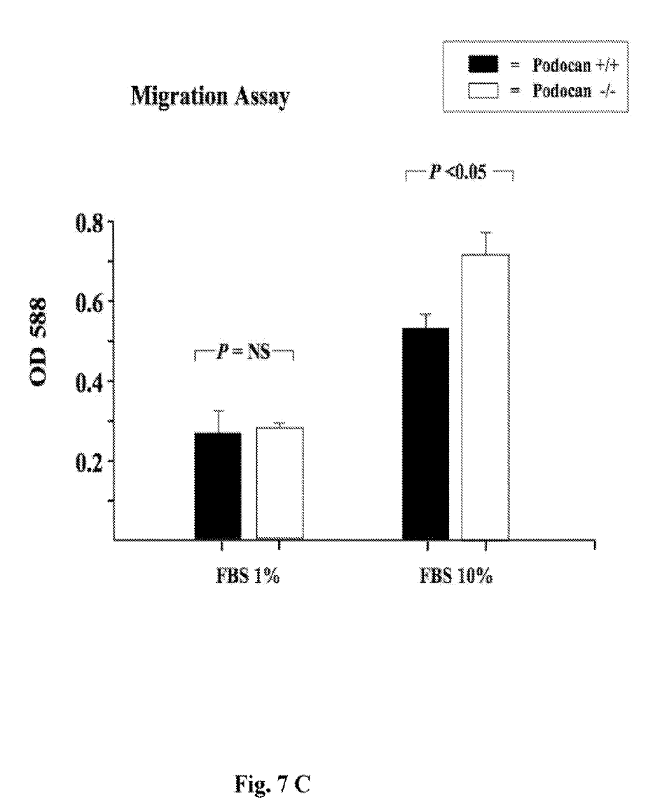

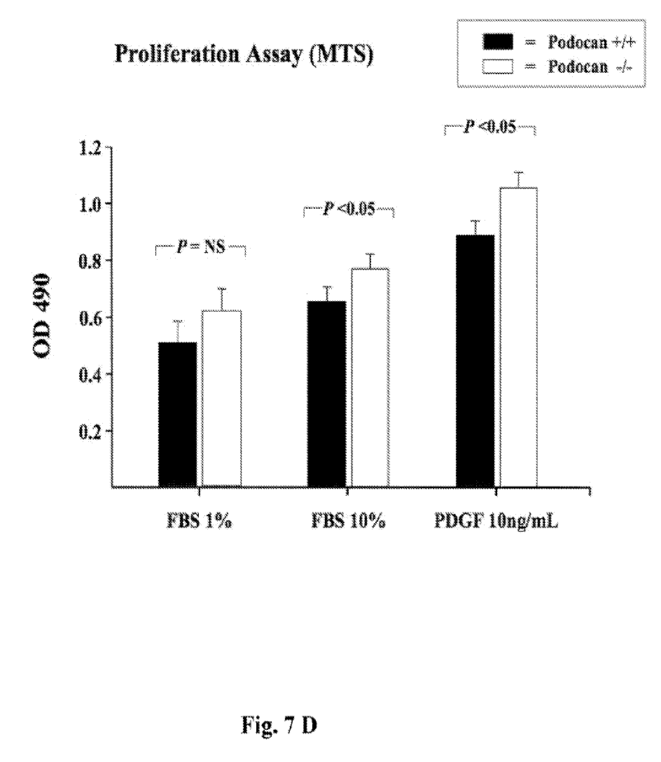

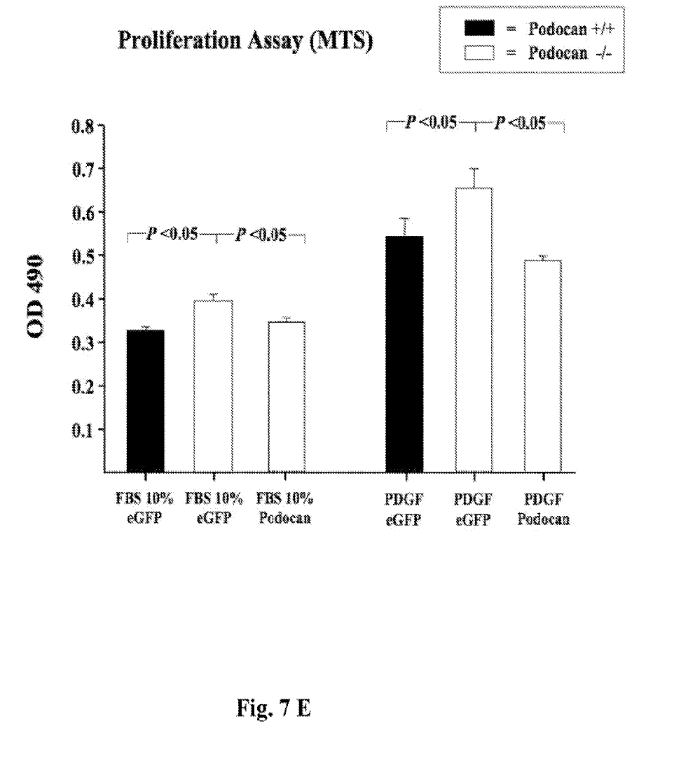

The migration of podocan deficient (−/−) and WT SMC was compared using a colorimetric cell migration assay based on the Boyden chamber principle. SMC were tested under low and intermediate serum conditions (1% and 10% FBS, respectively). Under low serum conditions there was no difference in transmigrated cells from podocan^{-/-} SMC when compared to WT (0.298±0.013 compared to 0.276±0.078; P=NS) (FIG. 7). However, with higher serum conditions, a significant increase in migratory activity was found in podocan^{-/-} SMC compared to WT (0.727±0.064 vs. 0.545±0.030, P<0.05) (FIG. 7).

The MTS assay (MTS stands for 3-(4,5-dimethylthiazol-2-yl)-5(3-carboxymethonyphenol)-2-(4-sulfophenyl)-2H-tetrazolium salt; the MTS assay is a colorimetric assay to determine cell proliferation (Promega)) was used to compare the proliferation of podocan-deficient and WT SMC in DMEM containing either 1% FBS, 10% FBS, in response to recombinant mouse PDGF (20 ng/ml). No significant difference in proliferative activity was found between podocan^{-/-} SMC and WT cells when cultured in 1% FBS (0.635±0.048 vs. 0.579±0.053, P=NS) (FIG. 7D). However, at 10% FBS and under PDGF stimulation a significant increase in proliferative activity was observed in podocan SMC compared to WT controls (10% FBS: 0.755±0.027 vs. 0.687±0.028, P<0.05; 20 ng/ml PDGF: 1.013±0.029 vs. 0.898±0.027, P<0.05) (FIG. 7D).

To test if the podocan^{-/-} SMC phenotype can be rescued by transfection with the WT podocan gene, WT-SMC (group 1) and podocan^{-/-} SMC (group 2) were transfected with eGFP serving as controls and were compared with podocan^{-/-} SMC transfected with podocan (group 3). Proliferation was measured in all 3 groups of transfected cells at 10% FBS (group1: 0.331±0.005; group2: 0.395±0.011; group3: 0.350±0.014) (group 1 vs. group 2, P<0.05 and group 2 vs. group 3, P<0.05) (FIG. 7E). A similar normalization of the podocan^{-/-} SMC phenotype was observed when repeating the experiment under 20 ng/ml PDGF stimulation (group 1: 0555±0.005; group 2: 0.652±0.019; group 3: 0.485±0.016) (group 1 vs. group 2, P<0.05 and group 2 vs. group 3, P<0.05) (FIG. 7E).

### Discussion.

To determine if the WT phenotype could be rescued, in vitro methods were utilized in order to explore SMC migration and proliferation of primary aortic SMC cultures. At baseline conditions under low serum medium, differences in migratory and proliferative activity between WT and −/− cells could not be detected (FIG. 7). However, when SMCs were cultured in 10% FBS containing media or when stimulated by PDGF, there was a significant increase in the migratory and proliferative activity of podocan^{-/-} cells when compared to WT (FIG. 7). These findings are also consistent with an accelerated outgrowth of SMC from aortic explants of podocan^{-/-} mice (FIGS. 7A and 7B)³¹. When podocan^{-/-} SMC were transfected with the WT podocan gene a complete normalization of the podocan^{-/-} SMC phenotype occurred with proliferative activity reduced to the WT level (FIG. 7).

The lack of difference in proliferation under low serum conditions, appears to be consistent with the in vivo finding that in quiescent cells, podocan is not expressed. Non-injured arterial walls do not express podocan and without injury there is no vascular phenotype in −/− mice at baseline. Unless the arterial wall SMC population is stimulated by an injury/repair signal and/or PDGF is abundantly expressed, the dramatic differences in SMC migratory and proliferative activity between the WT and podocan^{-/-} genotype do not become evident^{3,20,21}.

Taken together, the data set forth herein on the expression pattern of podocan in experimental and human arterial lesions and the effect of podocan genotype on SMC activation in vivo and in vitro demonstrates a highly selective negative feedback regulation of podocan on activated SMC occurring physiologically to limit arterial repair. In addition, no significant difference in the rate of luminal endothelial repair and re-endothelialization was found at any time point after arterial injury pointing to a selective regulatory effect of podocan on SMC and not on macro-vascular EC. In this context it is of particular interest that in human restenotic lesions as a classic example of exuberant arterial repair, increased migratory and proliferative activity of SMC have been described for many years by several groups^{3,19,20,32,33}. In human lesions retrieved by coronary atherectomy, podocan protein was nearly completely absent in SMC-rich human restenotic lesions, compared with a significant presence of podocan signals in primary human lesions with lesser SMC density. In contrast, no such selective relationship in symptomatic human coronary target lesions between SMC hyperplasia, paucity of podocan and abundance of PDGF has been described for other SLRP's such as Biglycan and Decorin in human atherosclerosis and in stented rabbit aorta^{13,34-37}.

### Method: Culture of SMC and Podocan Transfection

SMC were prepared by the explant method from aortas of −/− mice or wildtype littermates. Briefly, the aortas were freed of connective tissue and adherent perivascular fat, the endothelial cell layer of the intima was removed, and the arteries were cut into about 3-mm rectangular pieces. The pieces were placed in DMEM (Gibco) supplemented with 20% FBS, 100 U/ml penicillin, 100 g/ml streptomycin and 0.25 μg/ml amphotericin B (Cambrex) in a humidified atmosphere of 5% CO₂ and 95% air at 37° C. SMC exhibited a typical “hill and valley” growth pattern and the cell type was confirmed by morphological examination and smooth muscle alpha-actin staining (data not shown). Medium was replaced every other day. SMCs were serially passaged before reaching confluence, and all experiments were performed on SMC from passages 2 to 4. Cells were washed three times with HBSS and rendered quiescent in serum free DMEM for 24 hours prior to experiments. The expression vector encoding the full-length mouse podocan protein (pCDNA3.1-mPodocan) and control vector (pCDNA3.1) were transfected into smooth muscle cells using Fugene 6.0 (Roche). The cells were harvested at 48 h post-transfection for RNA and total protein extraction.

### Method: Cell Proliferation Assay

To evaluate the proliferation of podocan deficient (−/−) and WT SMCs cells (70% to 90% confluent) were trypsinized, washed 2× with PBS and added to gelatin-coated 96-well plates at a density of 5×10³ cells/well in DMEM containing either 1% FBS, 10% FBS, or recombinant mouse PDGF (R & D Systems). After culture for 72 hours, cell number was assessed using the MTS assay (Promega) with absorbance at 490 nm measured by spectrophotometry.

### Method: Cell Migration Assay

The migration of podocan deficient (−/−) and WT SMCs was examined using a colorimetric cell migration assay (Chemicon) based on the Boyden chamber principle using inserts with a pore size of 8 μm. SMCs were trypsinized, washed 2× with PBS, resuspended in 1% FBS in DMEM, and added to the top wells (2.5×10⁴ cells/300 μL). DMEM with 10% FBS or recombinant mouse PDGF (R & D Systems) was added to the bottom chamber. After 6 hours at 37° C., nonmigrating cells were scraped from the upper surface of the filter. Cells on the bottom surface were incubated with Cell Stain Solution (Chemicon), then subsequently extracted and detected by spectrophotometry (absorbance at 560 nm).

### Method: Statistical Analysis

For data analysis, the SPSS/PC+ software was used. Data are given as mean±SEM (in vivo data) and as mean±SD (in vitro data). After testing for normal distribution and equality of variances with Levene's F-test, the independent sample t-test was used to compare neointima formation, reendothelialization, SMC density, and arterial wall expression of Ki-67 in podocan^{-/-} and WT mice. Absorption at OD588 (migration assay) and OD490 (proliferation assay) were also compared using the independent sample t-test. Probability values were two-tailed and corrected for ties. P values <0.05 were considered significant.

### Example 4

### Podocan-Eluting Stents in a Porcine Model

Examples of the use and testing of a drug-eluting stent in a porcine animal model are set forth in Blindt et al. (A Novel Drug-Eluting Stent Coated with an Integrin-Binding Cyclic Arg-Gly-Asp Peptide Inhibits Neointimal Hyperplasia by Recruiting Endothelial Progenitor Cells; Interventional Cardiology (2006), Vol. 47, No. 9, 1786-1795) and Garcia-Touchard et al., (Zotarolimus-eluting stents reduce experimental coronary artery neointimal hyperplasia after 4 weeks, European Heart Journal, Volume 27 (2006), pp. 988-993).

Animal studies will be conducted in accordance with the standard guidelines for the care of laboratory animals. Porcine stent studies will be carried out much as described in, for example, Garcia-Touchard et al., Zotarolimus-eluting stents reduce experimental coronary artery neointimal hyperplasia after 4 weeks, European Heart Journal, Volume 27 (2006), pp. 988-993.

Stainless steel BiodivYsio coronary stents (3.0×15 mm; Abbott Laboratories) will be coated with a 10 μm thick polymer base layer and crimped onto delivery balloons (polymer-only stents) (for example, as described in Galli M, Bartorelli A, Bedogni F, DeCesare N, Klugmann S, Maiello L, Miccoli F, Moccetti T, Onofri M, Paolillo V, Pirisi R, Presbitero P, Sganzerla P, Viecca M, Zerbonii S, Lanteri G. Italian BiodivYsio open registry (BiodivYsio PC-coated stent): study of clinical outcomes of the implant of a PC-coated coronary stent. J Invasive Cardiol. 2000; 12:452-8). These polymer coated stents are loaded with podocan or podocan-inhibiting molecules by simple immersion in an alcoholic or aqueous or PBS-based solution of these compounds for 5 minutes, followed by an evaporation step at room temperature to dry the stent. The amount of podocan or podocan inhibiting molecules is controlled by varying the concentration of these compounds in the loading solution. Maximum loading doses are determined by the solubility of these compounds in a particular solvent system. The total loading dose in each setup will be confirmed/measured by sonication of the loaded stent in solvent to completely remove all of these compounds, followed by appropriate evaluation of the eluent measuring their respective concentrations. A minimum of five stents will be used per loading and elution study. (Lewis et al., Journal of Materials Science: Materials in Medicine 12 (2001) 865-870; Garcia-Touchard et al., European Heart Journal (2006) 27, 988-993; Shinozaki et al., Circ J 2005; 69: 295-300).

Three groups of podocan-eluting stents with escalating doses of podocan will be used:

Group 1 (10 μg/mm with total podocan load of 150 μg);

Group 2 (50 μg/mm with total podocan load of 750 μg);

Group 3 (100 μg/mm with total podocan load of 1500 μg);

To determine the total stent drug loading, five podocan eluting stents from each group will be each placed in 1.7 ml of acetonitrile/water (50:50) and sonicated for 1 h. The resulting solution will then be tested for the concentration of podocan by ELISA, determining the average load of podocan on these stents (expressed as weight in μg per stent length in mm). Stents will be sterilized with ethylene oxide and individually packaged and coded with a serial number.

One control stent (coated only with polymer) and one podocan-eluting stent will be implanted into the coronary arteries of 60 cross-bred juvenile swine, 2-4 months old, weighing 30 to 40 kg (n=20 animals per each podocan dose group). On the day of procedure, animals will be given oral aspirin 35 mg daily and cefazolin 200 mg twice per day. General anesthesia will be achieved by intra-muscular injection and ensuing intravenous infusion of ketamine 30 mg/kg and xylazine 3 mg/kg. Arterial access will be obtained by surgical cut down of the right external carotid artery and placement of an 8 F sheath, and an intra-arterial bolus of 10,000 units of heparin will be administered. Following guiding catheter access and angiography, two arteries will be selected based on size and visual suitability for stenting (length, straight segments, lack of large side branches). Usually the most appropriate vessels for stenting will be the left anterior descending artery (LAD) and the right coronary artery (RCA). Once the target arteries are selected, the randomization to podocan-eluting stents or control stents will be performed in a blinded fashion. The stent balloons will be inflated for less than 30 seconds to achieve a 1.1:1 to 1.2:1 stent-to-artery ratio. Following the procedure, the animals will be treated for the duration of the study with oral aspirin 325 mg daily and oral ticlopidine 250 mg twice daily. Future studies may include an arm in which animals are not given aspirin/ticlopidine treatment after the procedure in order to test the hypothesis that podocan therapy will diminish the need for post-intervention anti-platelet therapy.

After 28 days, the animals will be euthanized for histopathological examination and quantification. The hearts will be perfused overnight with 10% neutral buffered formalin at physiological pressure and embedded in paraffin. Sections 5 μm thick from the proximal and distal extra-stent segment and from the proximal, mid, and distal stented artery will be cut using a tungsten-carbide knife. The arterial tissues will subsequently be processed for (immuno)-histological studies and initially stained with haematoxylin-eosin and elastic van Gieson techniques.

Semi-quantitative histo-pathological evaluation will include vessel injury score (values of 0 for endothelium denuded, 1 for internal elastic lamina (IEL) lacerated, 2 for media lacerated, and 3 for external elastic lamina (EEL) lacerated), inflammation score (value of 0 for no inflammatory cells, 1 for mild inflammatory response but not circumferential, 2 for moderate to dense cellular aggregate but not circumferential, and 3 for circumferential dense cell infiltration of the struts), endothelialization score (0 for absent endothelium, 1 for present endothelium but <25% of luminal circumference, 2 for present endothelium between 25 and 75% of luminal circumference, and 3 for complete endothelialization), and hemorrhage, fibrin, luminal thrombus scores.

Quantitative morphometric measurements using digital planimetry (Image Pro Plus) will be performed to measure the cross-sectional area of lumen, IEL, EEL. Derived measurements include neointimal area (IEL area−lumen area) and percent area stenosis ((1-neointima area)×100).

### Example 5

### Podocan-Eluting Stents in a Rabbit Model of Aorto-Iliac Stenting

Animal studies will be conducted in accordance with the standard guidelines for the care of laboratory animals. Bilateral iliac arterial injury will be performed in New Zealand White (NZW) rabbits fed an atherogenic diet followed by stent implantation. Animals will be randomized to receive either podocan coated stents (group 1, 2, and 3) or bare metal stents (BMS) (control group) (Ribichini et al., Effects of Oral Prednisone After Stenting in a Rabbit Model of Established Atherosclerosis, Journal of the American College of Cardiology, Volume 50, Issue 2 (2007), pp. 176-185). Stented arterial segments will be harvested at 42 days and processed for (immuno)-histochemical analysis and in vitro analysis.

In detail, the experimental preparation of the NZW rabbits at an age of 3 to 4 months consists of feeding an atherogenic diet (1% cholesterol and 6% peanut oil, F4366-CHL, Bio-Serv, Inc, Frenchtown, N.J.) for 5 weeks to induce atherosclerosis (Ribichini et al., Effects of Oral Prednisone After Stenting in a Rabbit Model of Established Atherosclerosis, Journal of the American College of Cardiology, Volume 50, Issue 2 (2007), pp. 176-185).

Stainless steel BiodivYsio stents (Abbott Laboratories) will be coated with a 10 μm thick polymer base layer and crimped onto delivery balloons (polymer-only stents). Three groups of Podocan-eluting stents with escalating doses of podocan will be used:

Group 1 (10 μg/mm with total podocan load of 150 μg);

Group 2 (50 μg/mm with total podocan load of 750 μg);

Group 3 (100 μg/mm with total podocan load of 1500 μg);

To determine the total stent drug loading five podocan eluting stents from each group will be each placed in 1.7 ml of acetonitrile/water (50:50) and sonicated for 1 h. The resulting solution will then be tested for the concentration of podocan by ELISA determining the average load of podocan on these stents (expressed as weight in μg per stent length in mm) (Swanson N, Hogrefe K, Javed Q, Gershlick A H. In vitro evaluation of vascular endothelial growth factor (VEGF)-eluting stents. Int J Cardiol. 2003; 92:247-51). Stents will be sterilized with ethylene oxide and individually packaged and coded with a serial number.

Iliac arterial injury will be induced 1 week after start of atherogenic diet using a Fogarty catheter (3-F) as described previously (Farb A, Tang A L, Shroff S, Sweet W, Virmani R. Neointimal responses 3 months after (32)P beta-emitting stent placement. Int J Radiat Oncol Biol Phys. 2000; 48:889-98). Following balloon injury, the animals will be maintained on an atherogenic diet for 4 weeks. Subsequently, the diet will be switched to a low-cholesterol diet (containing 0.025%) until sacrifice.

In selected animals from each treatment group (n=4 stents), stented arteries will be explanted 7 days following deployment and perfused with ice-cold Ringer's lactate at physiologic pressure a (100 mmHg). The specimens will be harvested and immersed in fresh Dulbecco's modified Eagle's medium (DMEM) containing 0.1% bovine serum albumin (BSA). Vessels will be cut into stented and non-stented portions and put in serum-free media for 48 h (DMEM+0.1% BSA, 2 ml per well). The supernatant (4 ml from 2 wells per sample) will be transferred to clean Eppendorf tubes, spun at 1500 rpm at room temperature for 15 min, and the supernatant will be collected for the analysis of cytokines using Cytokine Array 1 (RayBiotech, Inc).

Rabbit aortic smooth muscle cells will be obtained from the American Type Culture Collection (Manassas, Va.) and maintained in rabbit aortic smooth muscle cell growth medium (Cell Applications, San Diego, Calif.).

Subsequently, SMC migration and proliferation studies will be performed as described above.

### Example 6

### Local Gene Transfer of Podocan Plasmid by Podocan Gene-Eluting Stents

This example tests the hypothesis that local delivery via a gene-eluting stent of naked plasmid DNA encoding for human podocan can achieve reductions in neointima formation without affecting reendothelialization.

Podocan plasmid (100 or 200 μg per stent)—coated BiodivYsio phosphorylcholine polymer stents and uncoated stents (bare metal stents or BMSs) will be deployed in a randomized, blinded fashion in iliac arteries of 40 normocholesterolemic and 16 hypercholesterolemic rabbits. Reendothelialization will be measured after 10 days and at 3 months. At 3 months, lumen cross-sectional area and percent cross-sectional narrowing will be examined by intra-vascular ultrasound and histopathologic analysis comparing podocan gene delivering stents with BMS in normo- and hyperlipidemic rabbits. Transgene expression in the vessel wall will be evaluated in the stented segments.

Method. Animals:

Animal studies will be conducted in accordance with the standard guidelines for the care of laboratory animals. New Zealand White (NZW) rabbits weighing 4.5 to 5 kg with iliac artery dimension of approximately 2.2 mm will be used. A subset of animals (n=16) will be placed on 1% cholesterol diet with 3% peanut oil for 4 weeks before initial intervention, which will be maintained throughout the follow up period as described (Farb A, Tang A L, Shroff S, Sweet W, Virmani R. Neointimal responses 3 months after (32)P beta-emitting stent placement. Int J Radiat Oncol Biol Phys. 2000; 48:889-98).

Method. Preparation of Podocan Plasmid Coated Stents:

The 15 mm BiodivYsio stent will be electro-polished, cleaned and coated with a phosphorylcholine polymer (PC) with or without podocan plasmid (100 or 200 μg per stent) under sterile conditions (Galli M, Bartorelli A, Bedogni F, DeCesare N, Klugmann S, Maiello L, Miccoli F, Moccetti T, Onofri M, Paolillo V, Pirisi R, Presbitero P, Sganzerla P, Viecca M, Zerboni S, Lanteri G. Italian BiodivYsio open registry (BiodivYsio PC-coated stent): study of clinical outcomes of the implant of a PC-coated coronary stent. J Invasive Cardiol. 2000; 12:452-8.; Whelan D M, van der Giessen W J, Krabbendam S C, van Vliet E A, Verdouw P D, Serruys P W, van Beusekom H M. Biocompatibility of phosphorylcholine coated stents in normal porcine coronary arteries. Heart. 2000; 83:338-45.) Coating and manufacturing will be performed by Biocompatibles UK Ltd, and the stent will be premounted on a 3-mm balloon catheter covered by a 5 F protection sleeve (as described in Galli et al. and Whelan et al., supra). The stents will be shipped at room temperature and be used within 6 months of manufacture. The stability and integrity of the plasmid will be verified by sequencing of DNA eluted from randomly selected stents. The podocan plasmid pcDNA3.1(+)-hPODN contains the human podocan coding sequence ((GenBank Locus BC030608)).

Method. In vivo Catheter Procedures and Intravascular Ultrasound Imaging Analysis:

After surgical exposure of the external carotid artery, a 5 F introduce sheath (Radifocus, Terumo) will be advanced to the lower abdominal aorta followed by administration of 1000 U heparin. Balloon denudation of the external iliac artery will be performed by sequential withdrawal (6 times) with a 2 F Fogarty balloon catheter (Baxter Edwards). Stent implantation will be performed by balloon inflation (20 seconds at 10 atm). In a subset of rabbits, stents will be implanted bilaterally. A single dose of aspirin 50 mg (Aspisol, Bayer) will be administered intravenously after procedure. For follow-up angiograms, the contralateral carotid artery will be exposed surgically.

To analyze the formation of intimal hyperplasia in vivo, intravascular ultrasound (IVUS) imaging will be performed at baseline and at 3 months follow-up using 2.5 F 40-Mhz transducers (Atlantis SR Plus, Boston Scientific Scimed) with motorize pull-back speed of 0.5 mm/s/Measurements will be made twice every 1 mm and will include in-stent area, lumen area, and intimal hyperplasia cross-sectional areas as published (Hoffmann R, Mintz G S, Dussaillant G R, Popma J J, Pichard A D, Satler L F, Kent K M, Griffin J, Leon M B. Patterns and mechanisms of in-stent restenosis. A serial intravascular ultrasound study. Circulation. 1996; 94:1247-54) and as described above.

Method. Histological and Ultrastructural Analysis:

Arterial specimens will be embedded in methyl methacrylate and cut with a diamond blade followed by metachromatic staining. For 6 different locations/serial sections, the extent of vessel injury quantified by the method of Schwartz et al. (Schwartz R S, Huber K C, Murphy J G, Edwards W D, Camrud A R, Vlietstra R E, Holmes D R. Restenosis and the proportional neointimal response to coronary artery injury: results in a porcine model. J Am Coll Cardiol. 1992; 19:267-74) inflammations core, and areas of neointima, media, native vessel lumen, and stent lumen will be measured as described above. Macrophages will be detected by RAM-11 and vascular smooth muscle cells by alpha-actin immuno-staining. In addition, reendothelialization will be measured by lectin staining and Evans Blue staining as described (Walter D H, Cejna M, Diaz-Sandoval L, Willis S, Kirkwood L, Stratford P W, Tietz A B, Kirchmair R, Silver M, Curry C, Wecker A, Yoon Y S, Heidenreich R, Hanley A, Kearney M, Tio F O, Kuenzler P, Isner J M, Losordo D W. Local gene transfer of phVEGF-2 plasmid by gene-eluting stents: an alternative strategy for inhibition of restenosis. Circulation. 2004; 110:36-45).

Method. Detection of Transgene Expression: RT-PCR:

RNA of whole-vessel segments will be extracted using the RNeasy Kit (Qiagen). cDNA synthesis will be performed with 1 μg of total RNA using the Superscript II kit (Life Technologies) and Advantage-GC cDNA polymerase (Clontech). For semi-quantification, QuantumRNA 18S internal standards will be used (Ambion). Reverse transcription-polymerase chain reaction (RT-PCR) products will be analyzed by 1% agarose gel electrophoresis.

Using specific primers for human podocan, a podocan-specific PCR product will be identified, and its detectability will be compared between extracts of rabbit iliac arteries from podocan gene-eluting stent treated groups, control group, and cultured rabbit smooth muscle cells.

### Method: Detection of Transgene Expression: In Situ Hybridization.

Human-podocan RNA expression will also be localized by in situ hybridization of frozen tissue-sections under RNase-free conditions. Sense or anti-sense riboprobes will be designed based on the above-mentioned primers using T7 RNA polymerase (Promega) and digoxigenin labeling (Roche). Hybridization of the riboprobes 920 ng/ml) will be performed at 55° C. for 18 hours. Vessel cross-sections will be incubated with sheep anti-DIG POD antibody (Roche) in TNB (100 mmol/L Tris HCl (pH 7.5), 150 mmol/L NaCl, 0.5% blocking reagent 1: 100 overnight at 4° C., followed by fluorescent CY3 at 1:50 in diluent from kit (TSA, Plus Cy3 System, Perkin Elmer). Slides will be mounted with Fluoromount G (Southern Biotech Associates), and red fluorescent reaction products will be visualized under fluorescent microscopy (Hutter R, Valdiviezo C, Sauter B V, Savontaus M, Chereshnev I, Carrick F E, Bauriedel G, Luderitz B, Fallon J T, Fuster V, Badimon J J. Caspase-3 and tissue factor expression in lipid-rich plaque macrophages: evidence for apoptosis as link between inflammation and atherothrombosis. Circulation. 2004; 109:2001-8).

Method. Podocan Protein in Arterial Tissue:

Tissue samples from rabbit iliac arteries will be homogenized in protein lysis buffer and samples will be used to measure podocan protein concentration. In addition, expression of human podocan in rabbit iliac artery will also be evaluated by immunofluorescence labeling using anti-human podocan antibody.

### Example 7

### The Effects of Podocan on Graft Vasculopathy

Given the data above demonstrating a distinct expression pattern of podocan in hyperplastic restenotic versus normo-cellular stable arterial lesions in human coronary arteries and the inhibitory effect of podocan on accelerated SMC migration and growth in podocan^{-/-} mice, in order to test the hypothesis that podocan expression is altered in GVP, the following experiments are set forth. These experiments will demonstrate that GVP results in a relative lack of the SMC-growth and migration inhibitory signal of podocan as described in detail for vascular SMC. Specifically, these tests evaluate the expression of podocan in arteries from transplanted hearts from patients with GVP in comparison with coronary arteries from patients without GVP. These experiments also evaluate podocan protein therapy in vivo in the setting of an in vivo model of GVP in wild-type mice (WT) and compare the extent of GVP in control treated animals after heterotopic cardiac transplant.

Evaluation of Podocan expression in Myocardial Biopsies of Cardiac Allografts and Normal Hearts

One hundred thirty-three endomyocardial biopsy samples from 11 cardiac allografts and 15 biopsy samples from 15 normal hearts will be analyzed for expression of podocan mRNA with quantitative RT-PCR. Myocardium from pre-transplantation normal donor hearts will be obtained from the right ventricle immediately after organ excision. Allograft myocardial biopsies will be obtained at routine follow-up biopsy after transplantation or when clinically indicated. At each cardiac catheterization, 5 biopsy samples will be obtained for histology to monitor allograft rejection, and 1 will be obtained and frozen for RNA extraction. Annual coronary angiography will be used to assess cardiac allograft vasculopathy (CAV). Coronary angiograms will be reviewed and compared with baseline angiograms independently by 2 cardiologists who will be unaware of the results of studies on podocan expression. CAV will be assessed according to the criteria established by Gao et al. (Gao S Z, Alderman E L, Schroeder J S, Silverman J F, Hunt S A. Accelerated coronary vascular disease in the heart transplant patient: coronary arteriographic findings. J Am Coll Cardiol. 1988; 12:334-40). CAV assessment will include the presence of focal stenosis, distal tapering or pruning, and loss or tertiary vessels. CAV will be assigned a numerical rating for severity as absent (0), mild (1), moderate (2), or severe (3).

### Isolation of RNA From Myocardial Biopsy Samples and RT-PCR

Total RNA will be isolated from myocardial biopsies using RNAzol B (Tel-Test) and will be used as template for cDNA synthesis. Primers assessing podocan expression and relevant cytokine expression will be used as described (Zerbe T, Uretsky B, Kormos R, Armitage J, Wolyn T, Griffith B, Hardesty R, Duquesnoy R. Graft atherosclerosis: effects of cellular rejection and human lymphocyte antigen. J Heart Lung Transplant. 1992; 11:S104-10; Zhao X M, Frist W H, Yeoh T K, Miller G G. Expression of cytokine genes in human cardiac allografts: correlation of IL-6 and transforming growth factor-beta (TGF-beta) with histological rejection. Clin Exp Immunol. 1993; 93:448-51; Zhao X M, Hu Y, Miller G G, Mitchell R N, Libby P. Association of thrombospondin-1 and cardiac allograft vasculopathy in human cardiac allografts. Circulation. 2001; 103:525-31; Zhao X M, Yeoh T K, Frist W H, Porterfield D L, Miller G G. Induction of acidic fibroblast growth factor and full-length platelet-derived growth factor expression in human cardiac allografts. Analysis by PCR, in situ hybridization, and immunohistochemistry. Circulation. 1994; 90:677-85; Ross et al., 2003). Quantitative RT-PCR will be performed with 32P-labeled dCTP to generate radioactively labeled PCR products. 4 PCR products will be run on 2% agarose gel, dried, and exposed to a PhosphorImager (Molecular Dynamics) for quantification. Standard curves within the exponential range of amplification for each gene will be generated with known amounts of cDNA template. The concentration of cDNA in each sample will be calculated from the standards run at the same time. The amount of cDNA for each gene will be normalized to the amount of cDNA of GAPDH, a constitutively expressed gene, in each sample. The ratio between each gene of interest and GAPDH will be used for comparison.

### Immunohistochemistry

Twenty specimens will be obtained from 3 explanted cardiac allografts during autopsy. Normal hearts will be obtained from patients who died of noncardiac diseases. Specimens will be fixed in 10% formalin and embedded in paraffin for processing. After deparaffinization, slides will be incubated sequentially in proteinase K/PBS (5 mg/mL; Boehringer Mannheim) for 20 minutes, 0.3% H₂O₂/PBS for 20 minutes, 5% horse serum/PBS for 1 hour, and first antibodies in 5% horse serum/PBS for 2 hours. The first antibodies used will be rabbit anti-human podocan, mouse anti-human smooth muscle alpha-actin (Sigma), and rabbit anti-human Ki-67 (DAKO). Rabbit or mouse IgG (Santa Cruz) will be used as first antibody in negative controls. After incubation and washing in PBS, slides will be incubated with biotinylated secondary antibodies (Vector) and developed with use of a Vectastain ABC kit (Vector) and a DAB substrate kit (Vector).

### Mouse Model of GVP:

### I. Animals

Animal studies will be conducted in accordance with the standard guidelines for the care of laboratory animals. Adult female B6.C-H2bm12 and wild-type C57BL/6 mice, 6-12 weeks old, will be purchased from Jackson Laboratories (Bar Harbor, Me.). The B6.C-H2bm12 and C57BL/6 strains differ at the I-A locus of MHC II but are identical at MHC I and minor MHC loci. Recombinant podocan was synthesized as described above. B6.C—H2bm12 strain donor hearts will be transplanted into wild-type C57BL/6 recipient mice. Intra-abdominal heterotopic heart transplantation will be performed using a modification of the method outlined by Corry et al. (Corry R J, Winn H J, Russell P S. Primarily vascularized allografts of hearts in mice. The role of H-2D, H-2K, and non-H-2 antigens in rejection. Transplantation. 1973; 16:343-50). Recipient mice in the podocan treatment group will receive daily recombinant podocan (200 microgram/kg in 200 microliter of phosphate-buffered saline [PBS]) intraperitoneally, beginning on postoperative day 0 (n=8). In the control group, the recipient mice will receive daily intraperitoneal PBS using a similar protocol (n=8). No immunosuppressant will be given. The donor hearts will be palpated daily to assess for acute rejection. The donor hearts will be harvested on day 24 after transplant. Previous studies have shown that the donor hearts in the control group reproducibly develop CAV within 24 days (Shi C, Russell M E, Bianchi C, Newell J B, Haber E. Murine model of accelerated transplant arteriosclerosis. Circ Res. 1994; 75:199-207).

### II. Morphometric Analysis

The explanted hearts will undergo serial sectioning (5-m thick) from the midventricular level to the base. Verhoeff elastic staining will be performed for morphometric analysis of arterial intimal lesions. All coronary arteries (diameter 30 to 350 μm in diameter) will be analyzed on a PC computer using the Image PRO PLUS software. Three cross sections of each mouse heart will be evaluated. The number of analyzed vessels per heart will number between 80 to 100. Luminal (L) and intimal areas (IL) will be traced and the areas quantitated the Image PRO PLUS software. Intimal thickening will be calculated according to the formula (Intima/IntimaLumen) and expressed as a percentage. The validity of this protocol has been previously reported (Armstrong A T, Strauch A R, Starling R C, Sedmak D D, Orosz C G. Morphometric analysis of neointimal formation in murine cardiac allografts. Transplantation. 1997; 63:941-7).

### III. Immunohistochemistry

The basal segments of explanted hearts will be used for immunohistochemical analysis. The primary antibodies used for immunohistochemistry will be as follows:

The first antibodies used will be rabbit anti-human, mouse anti-human smooth muscle alpha-actin (Sigma), and rabbit anti-human Ki-67 (DAKO). In addition, CD4 monoclonal antibodies (mAb) (clone L3T4), rat anti-mouse CD8a mAB (Ly-2; BD PharMingen, San Diego, Calif.), and rat anti-mouse MOMA-2 mAb for monocytes/macro-phages (Serotec, Raleigh, N.C.) will be used. Immunohistochemistry will be performed using the ABC immunoperoxidase technique. Perivascular and intimal regions will be graded by an observer blinded to the study design.

### IV. Graft-Infiltrating Cell Isolation and FACS Analysis

Hearts will be digested in collagenases-D solution. Isolated cells will be counted after lysis of erythrocytes. Labeling of cells will be performed by FITC- and PE-labeled CD4 and CD8 antibodies (BD PharMingen). Rabbit anti-mouse CXCR3 labeling will be followed with FITC-labeled goat anti-rabbit secondary Ab (Zymed). FACS analysis of labeled cells will be conducted on an EPICS XL-MCL flow cytometer (Coulter).

### V. Reverse Transcription Polymerase Chain Reaction

Total RNA will be isolated from donor hearts and recipient spleens using the trizol method (Invitrogen, San Diego, Calif.). RNA will be also isolated from 72-hr mixed leukocyte reactions (MLRs) using the RNeasy Mini Protocol™ (Qiagen, Valencia, Calif.) (Zhao X M, Frist W H, Yeoh T K, Miller G G. Expression of cytokine genes in human cardiac allografts: correlation of IL-6 and transforming growth factor-beta (TGF-beta) with histological rejection. Clin Exp Immunol. 1993; 93:448-51). Two micrograms of DNase I treated RNA will then be used to synthesize the first strand of cDNA by the SuperScript First-Strand Synthesis System (Invitrogen). TaqMan-based PCR assays will be used to measure DNA using an ABI Prism 770 Sequence Detection System (Applied Biosystems, Foster City, Calif.). A master mix will be used consisting of 12.5 L of iTaq SYBR-Green Supermix with Rox (BioRad, CA), 1 L of 20 M forward primer, 1 L of 20 M reverse primer, and sterile water.

The cDNA product will be amplified using PCR primers specific for mouse podocan. PCR conditions will be 95° C. for 3 min, 40 cycles of 95° C. for 10 s, 64° C. for 30 s, and 72° C. for 20 s. All qtPCR assays will contain no-template control samples (negative controls) and five samples consisting of mouse genomic DNA added to reactions in duplicate to produce standards. The threshold cycle values from the genomic DNA standards will be used to create a standard curve to assess the amount of DNA in samples. All samples will be run in duplicate. Data will be reported as quantity of transcript (as reported by Ct) per 2 g of RNA.

### VI. Mixed Leukocyte Reaction

In order to confirm that any reductions in intimal hyperplasia with podocan treatment are SMC-mediated and not mediated by immunomodulation we evaluated whether podocan has any direct effect on lymphocyte biology. A total of 8105 splenocyte responder cells (C57BL/6) will be incubated with similar number of irradiated stimulator cells (B6.C-H2bm12) for 72 hr, followed by pulsing with 0.5Ci of [3H] thymidine (Amersham, Cleveland, Ohio) for 14 hours (Shi C, Lee W S, He Q, Zhang D, Fletcher D L, Jr., Newell J B, Haber E. Immunologic basis of transplant-associated arteriosclerosis. Proc Natl Acad Sci USA. 1996; 93:4051-6; Yun J J, Whiting D, Fischbein M P, Banerji A, Irie Y, Stein D, Fishbein M C, Proudfoot A E, Laks H, Berliner J A, Ardehali A. Combined blockade of the chemokine receptors CCR1 and CCR5 attenuates chronic rejection. Circulation. 2004; 109:932-7). The cells will be harvested with a semi-automated cell harvester and counted on a beta scintillation counter. Exogenous podocan will be added to each well at varying concentrations at the start of the mixed leukocyte reaction (MLR) to assess direct immunomodulatory properties of podocan. All MLRs will be performed in triplicate, and repeated three times (using three animals).

### VII. ELISPOT

In order to confirm that any reductions in intimal hyperplasia with podocan treatment are SMC-mediated and not mediated by immunomodulation we evaluated cells for the presence of IFN via ELISPOT. ELISPOT assays for murine IFN- will be performed according to the manufacturer's guidelines (BD Biosciences, San Diego, Calif.). In brief, 400,000 cells from a 48-hr MLR will be placed on plates that had been previously coated with a goat anti-murine IFN-antibody for 24 hr. The wells will then be washed and reacted with a biotinylated goat antimurine IFN-antibody. The spots will be visualized with 3-amino-9-ethylcarbazole chromogen (Sigma-Aldrich, St. Louis, Mo.). Visualization and analysis will be performed using Immunospot Series 1 Analyzer (Cellular Technology, Cleveland, Ohio). All assays will be performed in triplicate and will be repeated three times.

### Example 8

### The Effect of Podocan on Pulmonary Arterial Hypertension (PAH)

Given the data above demonstrating a distinct expression pattern of podocan in hyperplastic restenotic versus normo-cellular stable arterial lesions in human coronary arteries we postulate a possible alteration of podocan expression in PAH. This altered podocan expression will result in a relative lack of the SMC-growth and migration inhibitory signal of podocan, as described in detail for vascular SMC (SMC). Specifically, these tests evaluate the expression of podocan in pulmonary arteries from patients with PAH in comparison with pulmonary arteries from patients without PAH. These experiments also evaluate the expression of podocan in SMC cultured from pulmonary arteries (PASMC) from patients with PAH in comparison with pulmonary arteries from patients without PAH and to correlate podocan expression with migratory and proliferative activity of these cells. Further, these experiments test the treatment of PASMC derived from patients with PAH and control lungs with escalating doses of podocan protein and with podocan plasmid and evaluate the effect of podocan on PASMC migration and proliferation. Finally, these tests evaluated the effects of podocan overexpression in vivo in the setting of an in vivo model of PAH in wild-type (WT) rats and wild-type mice (WT) and compare the extent of PAH after monocrotaline treatment in podocan WT and podocan KO (−/− podocan genotype) mice.

Evaluation of Podocan Expression in Lesions from Patients with PAH:

Lung tissue from patients with pulmonary hypertension and control subjects will be obtained from the pulmonary hypertension tissue bank of Mount Sinai hospital. Samples will be obtained from patients with familial PAH (n=6) and idiopathic PAH (n=6). These patients will have received heart-lung transplantation for pulmonary arterial hypertension. Control lung (n=6) samples are comprised of tissue taken from the uninvolved lobe after pneumonectomy for lung neoplasia or from unused donors. All subjects or their relatives will give informed written consent, and the study will have approval from the Local Research Ethics Committee. Formalin-fixed, paraffin-embedded lung sections (5 μm) will be processed using pretreatment with 0.1% Saponin for 15 minutes as antigen retrieval technique. Sections will be stained with anti-podocan antibodies, anti-ki67 (Dako) and smooth muscle alpha-actin (Sigma). The extent of podocan expression in the smooth muscle and extra-cellular matrix of normal and hypertensive arteries (100 to 200 μm diameter) will be determined by counting the total number of smooth muscle cell nuclei and the number of nuclei, which stain positively for Ki-67, including at least 10 arteries from each case. The percentage of Ki-67 positive nuclei will then be calculated for controls, IPAH, and FPAH cases. The percentage of cells whose cytoplasm stained positively for podocan will be calculated similarly.

Evaluation of Podocan Expression in Human PASMC from Patients with Primary PAH and from Patients without PAH

Proximal and peripheral segments of human pulmonary artery will be obtained from unused donors (n=5) for transplantation. The Mount Sinai School ethical review committees will have approved the study, and subjects or relatives will have given informed written consent. Pulmonary artery smooth muscle cells (PASMCs) will be explanted from proximal lobar arteries and peripheral arteries (1 to 2 mm external diameter), as previously described (Wharton J, Davie N, Upton P D, Yacoub M H, Polak J M, Morrell N W. Prostacyclin analogues differentially inhibit growth of distal and proximal human pulmonary artery smooth muscle cells. Circulation. 2000; 102:3130-6). Cells will be maintained in 10% fetal bovine serum (FBS)/Dulbecco's modified Eagle Medium (DMEM) and will be used between passage 4 and 6. The smooth muscle phenotype of isolated cells will be confirmed by positive immuno-fluorescence with antibodies to smooth muscle actin (IA4), smooth muscle specific myosin (hsm-v), fibronectin, and vimentin, as described (Morrell N W, Upton P D, Kotecha S, Huntley A, Yacoub M H, Polak J M, Wharton J. Angiotensin II activates MAPK and stimulates growth of human pulmonary artery smooth muscle via AT1 receptors. Am J Physiol. 1999; 277:L440-8). In these cultured cells, the expression of podocan will be assessed at the mRNA and protein level by RT-PCR and Western blotting. In addition, protein expression will be differentiated between cytoplasmic expression and synthesis of secreted podocan measurable in cell culture supernatant by ELISA and the results will be compared for familial PAH (n=6) and idiopathic PAH (n=6) from patients that had received heart-lung transplantation for pulmonary arterial hypertension as well as control lung (n=6) obtained from the uninvolved lobe after pneumonectomy for lung neoplasia or from unused donors.

### Evaluation of Podocan Treatment Effects on Human PASMC in Culture

After establishing the intrinsic degree of podocan expression in PASMCs from familial PAH (n=6) and idiopathic PAH (n=6) from patients that had received heart-lung transplantation for pulmonary arterial hypertension as well as control lung (n=6) obtained from the uninvolved lobe after pneumonectomy for lung neoplasia or from unused donors, the effect of treatment with escalating doses of recombinant podocan will be tested.

The following doses of recombinant podocan will be given to these cells in culture

Group 1 (10 μg/ml);

Group 2 (100 μg/ml);

Group 3 (500 μg/ml);

All 3 groups will be compared to untreated control PASMC. Migratory and proliferative activity of PASMCs will be evaluated as described above. Using the same methodology, PASMCs treated with a plasmid containing the human podocan sequence will be evaluated and compared to PASMCs treated with plasmid containing only an eGFP sequence. In addition, modulation of the expression of podocan after plasmid treatment will be verified by RT-PCR as described above.

### Mouse and Rat Model of Monocrotaline-Induced Pulmonary Hypertension:

Animal studies will be conducted in accordance with the standard guidelines for the care of laboratory animals.

Mouse model of PAH:

Monocrotaline (MCT) (Sigma-Aldrich) will be converted to MCTp as previously published (Raoul W, Wagner-Ballon O, Saber G, Hulin A, Marcos E, Giraudier S, Vainchenker W, Adnot S, Eddahibi S, Maitre B. Effects of bone marrow-derived cells on monocrotaline- and hypoxia-induced pulmonary hypertension in mice. Respir Res. 2007; 8:8). CTp will be dissolved in N,N-dimethylformamide (DMF/RPMI 1640) just before use. Animals will be anesthesized with isoflurane (Forene, Abbott) and given a single injection of MCTp at a dose of 5 mg/kg into the jugular vein. As published (Raoul et al.), this treatment will be followed within 15 days by moderate pulmonary inflammation and remodeling of the small distal pulmonary vessels. These experiments will be done in WT and podocan −/− mice.

Mice (WT and podocan −/−) will be euthanized 15 days after initial MCTp treatment. Right ventricular systolic pressure (RVSP) is measured prior to euthananization and subsequently the right ventricle/left ventricle/septum weight ratio (RV/LV+S) will be measured as described (Raoul et al.). In addition, after perfusion fixation and paraffin embedding 4 μm thick lung sections will be cut and stained with hematoxylin-eosin. In each mouse 30 intra-acinar vessels accompanying alveolar ducts or alveoli will be morphometrically examined by an observer blinded to the study design.

### REFERENCES

1. Fuster V, Moreno P R, Fayad Z A, Corti R, Badimon J J. Atherothrombosis and high-risk plaque: part I: evolving concepts. J Am Coll Cardiol. 2005; 46:937-54.
2. Ross R. Atherosclerosis is an inflammatory disease. Am Heart J. 1999; 138:S419-20.
3. Ip J H, Fuster V, Badimon L, Badimon J, Taubman M B, Chesebro J H. Syndromes of accelerated atherosclerosis: role of vascular injury and smooth muscle cell proliferation. J Am Coll Cardiol. 1990; 15:1667-87.
4. Abraham L C, Dice J F, Lee K, Kaplan D L. Phagocytosis and remodeling of collagen matrices. Exp Cell Res. 2007; 313:1045-55.
5. Catania J M, Chen G, Parrish A R. Role of matrix metalloproteinases in renal pathophysiologies. Am J Physiol Renal Physiol. 2007; 292:F905-11.
6. Eikmans M, Baelde J J, de Heer E, Bruijn J A. ECM homeostasis in renal diseases: a genomic approach. J Pathol. 2003; 200:526-36.
7. Mancini A, Di Battista J A. Transcriptional regulation of matrix metalloprotease gene expression in health and disease. Front Biosci. 2006; 11:423-46.
8. Ruiz-Torres M P, Lopez-Ongil S, Griera M, Diez-Marques M L, Rodriguez-Puyol M, Rodriguez-Puyol D. The accumulation of extracellular matrix in the kidney: consequences on cellular function. J Nephrol. 2005; 18:334-40.
9. Iozzo R V. The biology of the small leucine-rich proteoglycans. Functional network of interactive proteins. J Biol Chem. 1999; 274:18843-6.
10. Ameye L, Young M F. Mice deficient in small leucine-rich proteoglycans: novel in vivo models for osteoporosis, osteoarthritis, Ehlers-Danlos syndrome, muscular dystrophy, and corneal diseases. Glycobiology. 2002; 12:107R-16R.
11. Bi Y, Nielsen K L, Kilts T M, Yoon A, M A K, Wimer H F, Greenfield E M, Heegaard A M, Young M F. Biglycan deficiency increases osteoclast differentiation and activity due to defective osteoblasts. Bone. 2006; 38:778-86.
12. Kolb M, Margetts P J, Sime P J, Gauldie J. Proteoglycans decorin and biglycan differentially modulate TGF-beta-mediated fibrotic responses in the lung. Am J Physiol Lung Cell Mol. Physiol. 2001; 280:L1327-34.
13. Nili N, Cheema A N, Giordano F J, Barolet A W, Babaei S, Hickey R, Eskandarian M R, Smeets M, Butany J, Pasterkamp G, Strauss B H. Decorin inhibition of PDGF-stimulated vascular smooth muscle cell function: potential mechanism for inhibition of intimal hyperplasia after balloon angioplasty. Am J Pathol. 2003; 163:869-78.
14. Schaefer L, Beck K F, Raslik I, Walpen S, Mihalik D, Micegova M, Macakova K, Schonherr E, Seidler D G, Varga G, Schaefer R M, Kresse H, Pfeilschifter J. Biglycan, a nitric oxide-regulated gene, affects adhesion, growth, and survival of mesangial cells. J Biol Chem. 2003; 278:26227-37.
15. Ross M D, Bruggeman L A, Hanss B, Sunamoto M, Marras D, Klotman M E, Klotman P E. Podocan, a novel small leucine-rich repeat protein expressed in the sclerotic glomerular lesion of experimental HIV-associated nephropathy. J Biol Chem. 2003; 278:33248-55.
16. Shimizu-Hirota R, Sasamura H, Kuroda M, Kobayashi E, Saruta T. Functional characterization of podocan, a member of a new class in the small leucine-rich repeat protein family. FEBS Lett. 2004; 563:69-74.
17. Hutter R, Carrick F E, Valdiviezo C, Wolinsky C, Rudge J S, Wiegand S J, Fuster V, Badimon J J, Sauter B V. Vascular endothelial growth factor regulates reendothelialization and neointima formation in a mouse model of arterial injury. Circulation. 2004; 110:2430-5.
18. Hutter R, Valdiviezo C, Sauter B V, Savontaus M, Chereshnev I, Carrick F E, Bauriedel G, Luderitz B, Fallon J T, Fuster V, Badimon J J. Caspase-3 and tissue factor expression in lipid-rich plaque macrophages: evidence for apoptosis as link between inflammation and atherothrombosis. Circulation. 2004; 109:2001-8.
19. Bauriedel G, Windstetter U, DeMaio S J, Jr., Kandolf R, Hofling B. Migratory activity of human smooth muscle cells cultivated from coronary and peripheral primary and restenotic lesions removed by percutaneous atherectomy. Circulation. 1992; 85:554-64.
20. Hart C E, Clowes A W. Platelet-derived growth factor and arterial response to injury. Circulation. 1997; 95:555-6.
21. Jawien A, Bowen-Pope D F, Lindner V, Schwartz S M, Clowes A W. Platelet-derived growth factor promotes smooth muscle migration and intimal thickening in a rat model of balloon angioplasty. J Clin Invest. 1992; 89:507-11.
22. Formato M, Farina M, Spirito R, Maggioni M, Guarino A, Cherchi G M, Biglioli P, Edelstein C, Scanu A M. Evidence for a proinflammatory and proteolytic environment in plaques from endarterectomy segments of human carotid arteries. Arterioscler Thromb Vasc Biol. 2004; 24:129-35.
23. Clowes A W, Berceli S A. Mechanisms of vascular atrophy and fibrous cap disruption. Ann N Y Acad. Sci. 2000; 902:153-61; discussion 161-2.
24. Hutter R, Sauter B V, Reis E D, Roque M, Vorchheimer D, Carrick F E, Fallon J T, Fuster V, Badimon J J. Decreased reendothelialization and increased neointima formation with endostatin overexpression in a mouse model of arterial injury. Circulation. 2003; 107:1658-63.
25. Leclair R J, Durmus T, Wang Q, Pyagay P, Terzic A, Lindner V. Cthrc1 Is a Novel Inhibitor of Transforming Growth Factor-{beta} Signaling and Neointimal Lesion Formation. Circ Res. 2007.
26. Lee R T, Yamamoto C, Feng Y, Potter-Perigo S, Briggs W H, Landschulz K T, Turi T G, Thompson J F, Libby P, Wight T N. Mechanical strain induces specific changes in the synthesis and organization of proteoglycans by vascular smooth muscle cells. J Biol Chem. 2001; 276:13847-51.
27. Mallawaarachchi C M, Weissberg P L, Siow R C. Smad7 gene transfer attenuates adventitial cell migration and vascular remodeling after balloon injury. Arterioscler Thromb Vasc Biol. 2005; 25:1383-7.
28. Reis E D, Roque M, Cordon-Cardo C, Drobnjak M, Fuster V, Badimon J J. Apoptosis, proliferation, and p27 expression during vessel wall healing: time course study in a mouse model of transluminal femoral artery injury. J Vasc Surg. 2000; 32:1022-9.
29. Kim W H, Hong M K, Virmani R, Kornowski R, Jones R, Leon M B. Histopathologic analysis of in-stent neointimal regression in a porcine coronary model. Coron Artery Dis. 2000; 11:273-7.
30. Stadius M L, Gown A M, Kernoff R, Schwartz S M. Does sequential balloon injury of an artery lead to a different outcome than a single injury? An experimental study of angioplasty. Coron Artery Dis. 1996; 7:247-55.
31. Kenagy R D, Hart C E, Stetler-Stevenson W G, Clowes A W. Primate smooth muscle cell migration from aortic explants is mediated by endogenous platelet-derived growth factor and basic fibroblast growth factor acting through matrix metalloproteinases 2 and 9. Circulation. 1997; 96:3555-60.
32. O'Brien E R, Alpers C E, Stewart D K, Ferguson M, Tran N, Gordon D, Benditt E P, Hinohara T, Simpson J B, Schwartz S M. Proliferation in primary and restenotic coronary atherectomy tissue. Implications for antiproliferative therapy. Circ Res. 1993; 73:223-31.
33. O'Brien E R, Urieli-Shoval S, Garvin M R, Stewart D K, Hinohara T, Simpson J B, Benditt E P, Schwartz S M. Replication in restenotic atherectomy tissue. Atherosclerosis. 2000; 152:117-26.
34. Kunjathoor V V, Chiu D S, O'Brien K D, LeBoeuf R C. Accumulation of biglycan and perlecan, but not versican, in lesions of murine models of atherosclerosis. Arterioscler Thromb Vasc Biol. 2002; 22:462-8.
35. O'Brien K D, Olin K L, Alpers C E, Chiu W, Ferguson M, Hudkins K, Wight T N, Chait A. Comparison of apolipoprotein and proteoglycan deposits in human coronary atherosclerotic plaques: colocalization of biglycan with apolipoproteins. Circulation. 1998; 98:519-27.
36. Shimizu-Hirota R, Sasamura H, Kuroda M, Kobayashi E, Hayashi M, Saruta T. Extracellular matrix glycoprotein biglycan enhances vascular smooth muscle cell proliferation and migration. Circ Res. 2004; 94:1067-74.
37. Yamakawa T, Bai H Z, Masuda J, Sawa Y, Shirakura R, Ogata J, Matsuda H. Differential expression of proteoglycans biglycan and decorin during neointima formation after stent implantation in normal and atherosclerotic rabbit aortas. Atherosclerosis. 2000; 152:287-97.
38. Joner M, Finn A V, Farb A, Mont E K, Kolodgie F D, Ladich E, Kutys R, Skorija K, Gold H K, Virmani R. Pathology of drug-eluting stents in humans: delayed healing and late thrombotic risk. J Am Coll Cardiol. 2006; 48:193-202.
39. Nilsen D W, Melberg T, Larsen A I, Barvik S, Bonarjee V. Late complications following the deployment of drug eluting stents. Int J Cardiol. 2006; 109:398-401.
40. Ribichini et al., Effects of Oral Prednisone After Stenting in a Rabbit Model of Established Atherosclerosis, Journal of the American College of Cardiology, Volume 50, Issue 2 (2007), pp. 176-185
41. Koren et al., Efficient transduction and seeding of human endothelial cells onto metallic stents using bicistronic pseudo-typed retroviral vectors encoding vascular endothelial growth factor, Cardiovascular Revascularization Medicine, Volume 7, Issue 3 (2006), pp. 173-178
42. Swanson et al., In vitro evaluation of vascular endothelial growth factor (VEGF)-eluting stents, International Journal of Cardiology, Volume 92 (2003), pp. 265-269
43. Stefanadis et al., Inhibition of plaque neovascularization and intimal hyperplasia by specific targeting vascular endothelial growth factor with bevacizumab-eluting stent: An experimental study, Atheroschlerosis, Mar. 21, 2007
44. Walter, et al., Local Gene Transfer of phVEGF-2 Plasmid by Gene-Eluting Stents: An Alternative Strategy for Inhibition of Restenosis, Circulation: The Journal of the American Heart Association, Volume 110 (2004), pp. 36-45
45. Garcia-Touchard et al., Zotarolimus-eluting stents reduce experimental coronary artery neointimal hyperplasia after 4 weeks, European Heart Journal, Volume 27 (2006), pp. 988-993
46. Hutter et al., Evidence for Increased Neointima Formation in Podocan Knock-out Mice compared to C57/BL6 Wild Type Mice after Arterial Denudating Injury; The Mount Sinai Journal of Medicine, (May 2006) Vol. 73, No. 3, 637
47. McEwan et al., Structural Correlations in the Family of Small Leucine-Rich Repeat Proteins and Proteoglycans; Journal of Structural Biology, Vol. 155 (2006), 294-305
48. Blindt et al., A Novel Drug-Eluting Stent Coated with an Integrin-Binding Cyclic Arg-Gly-Asp Peptide Inhibits Neointimal Hyperplasia by Recruiting Endothelial Progenitor Cells; Interventional Cardiology (2006), Vol. 47, No. 9, 1786-1795
49. Hutter et al., Evidence for Increased Neointima Formation in Podocan Knock-out Mice compared to C57/BL6 Wild Type Mice after Arterial Denudating Injury; oral presentation at American Heart Association conference Nov. 15, 2006 (poster slides and write-up of presentation)
50. WO 2006/132,788 (Byers-Horner et al.)
51. WO 2004/075,781 (Carpenter et al.)
52. U.S. Pat. No. 6,818,016 (Nabel et al.)
53. Newby A C, Zaltsman A B. Molecular mechanisms in intimal hyperplasia. J Pathol. 2000; 190:300-9.

1. A method of treating occlusion of a body vessel which comprises administering podocan or a functional equivalent that regulates smooth muscle cell activity.

2. The method of claim 1, wherein podocan or the functional equivalent thereof is linked to or embedded in a matrix or other peptide/protein.

3. The method of claim 1, wherein body vessel is a blood vessel.

4. The method of claim 1, wherein the smooth muscle cell activity is smooth muscle cell proliferation or smooth muscle cell migration.

5. The method of claim 1, wherein the occlusion is caused by a condition selected from the group consisting of atherosclerosis, restenosis of a blood vessel, transplant vasculopathy, vein-graft atherosclerosis, thrombosis, angioplasty restenosis, and pulmonary hypertension.

6. The method of claim 1, which comprises administering locally.

7. The method of claim 6, which comprises locally injecting podocan.

8. The method of claim 5, wherein the condition comprises pulmonary hypertension and podocan or a functional equivalent is administered through a right-heart luminal device in the pulmonary artery.

9. The method of claim 6, which comprises locally administering podocan or the functional equivalent by placing a medical or biocompatible device coated with podocan protein or its functional equivalent at the site of the occlusion.

10. The method of claim 6, which comprises locally administering podocan or the functional equivalent by placing a medical or biocompatible device coated with a nucleic acid encoding podocan or its functional equivalent at the site of the occlusion.

11. The method of claim 9, wherein the medical or biocompatible device is an intraluminal device.

12. The method of claim 11, wherein the intraluminal device is selected from the group consisting of a stent, a wire, a catheter, or a sheath.

13. The method of claim 12, wherein the intraluminal device is a stent.

14. The method of claim 1, wherein said body vessel is selected from the group consisting of the artery, vein, common bile duct, pancreatic duct, kidney duct, esophagus, trachea, urethra, bladder, uterus, ovarian duct, fallopian tube, vas deferens, prostatic duct, or lymphatic duct.

15. The method of claim 1 wherein podocan or the functional equivalent is administered in combination with a compound that inhibits proliferation of smooth muscle cells.

16. The method of claim 15 wherein said compound is selected from paclitaxel, rapamycin, actinomycin D, or radioactivity.

17. A method for diagnosing atherosclerosis in a patient, which comprises (i) obtaining a sample from said patient, (ii) measuring an expression level of podocan in said sample (iii) comparing said expression level with a standard, and wherein an increase in the level of podocan as compared to the podocan standard indicates atherosclerosis.

18. The method according to claim 17 wherein said measuring step comprises determining the expression level of podocan polypeptide or podocan mRNA.

19. The method of claim 1, wherein podocan or the functional equivalent is provided with a cell penetrating peptide.

20. The method according to claim 1, which comprises combining podocan or the functional equivalent with a homing peptide.

21. An intraluminal device coated with a nucleic acid encoding podocan or a functional equivalent of podocan.

22. An intraluminal device coated with a podocan polypeptide or a functional equivalent of said polypeptide.

23. The device of claim 21 wherein the intraluminal device is selected from the group consisting of a stent, a wire, a catheter, or a sheath.

24. The device of claim 21 wherein the intraluminal device is further coated with collagen or a collagen matrix.

25. The device of claim 24, wherein the nucleic acid encoding podocan or a functional equivalent of podocan is embedded in the collagen or collagen matrix.

26. A method of treating occlusion of a body vessel by administering an agent that regulates smooth muscle cell activity, wherein said agent is a podocan inhibitor or a functional equivalent thereof.

27. The method of claim 26 wherein the occlusion comprises a vulnerable plaque.

28. The method of claim 26 wherein the inhibitor is a member selected from the group consisting of a podocan antisense oligonucleotide, a podocan-specific RNAi construct, a podocan antibody or a small molecule inhibitor of podocan.

29. A method of inhibiting smooth muscle cell proliferation which comprises contacting a smooth muscle cell with podocan or a functional equivalent thereof; whereby proliferation of said smooth muscle cell is inhibited.

30. The method of claim 29 wherein said smooth muscle cell comprises a vascular smooth muscle cell.

31. The method of claim 29, wherein said contacting comprises administering to a site at risk of undesired smooth muscle cell proliferation a cell growth inhibitory amount of podocan or a functional equivalent thereof, whereby a smooth muscle cell proliferative disorder is treated.

32. The method of claim 29, wherein said contacting comprises administering to a patient at risk of restenosis an effective amount of podocan or a functional equivalent thereof for inhibiting vascular smooth muscle cell proliferation.

33. The method of claim 32 which comprises administering an effective amount of podocan or a functional equivalent thereof to said patient before, during or after an angioplasty procedure.

34. The method of claim 33 wherein said administering includes delivering podocan or a functional equivalent thereof to an angioplasty site in said patient.

35. The method of claim 33 wherein said angioplasty procedure includes placing a stent at an angioplasty site in said patient.

36. The method of claim 35 wherein said stent is a drug-eluting stent capable of releasing podocan or a functional equivalent thereof in situ.

37. The method of claim 34 wherein said contacting comprises administering podocan or a functional equivalent thereof to a patient at risk of atherosclerosis progression whereby the risk of atherosclerosis progression in the patient is treated.

38. The method of claim 29 wherein said contacting comprises administering podocan or a functional equivalent thereof to a patient at risk of keloid formation.

39. The method of claim 29 wherein said contacting comprises administering podocan or a functional equivalent thereof to a patient suffering from cancer originating from a smooth muscle cell, whereby proliferation of a cancer cell is inhibited.

40. The method of claim 29 which comprises combining podocan or its functional equivalent with a cell penetrating peptide.

41. The method according to claim 29, which comprises combining podocan or its functional equivalent with a homing peptide.

42. The method of claims 10, wherein the medical or biocompatible device is an intraluminal device.

43. The method of claim 42, wherein the intraluminal device is selected from the group consisting of a stent, a wire, a catheter, or a sheath.

44. The method of claim 43, wherein the intraluminal device is a stent.

45. The device of claim 22 wherein the intraluminal device is selected from the group consisting of a stent, a wire, a catheter, or a sheath.

46. The device of claim 22 wherein the intraluminal device is further coated with collagen or a collagen matrix.

47. The device of claim 46, wherein the podocan polypeptide or a functional equivalent of said polypeptide is embedded in the collagen or collagen matrix.
